SEQUENCE LISTING (52 sequences)

SEQ ID NO: 1 - protein, 23 aa, Homo sapiens
GKVLLLFQPNELQAMVIGNTNYD

SEQ ID NO: 2 - protein, 15 aa, Homo sapiens
LLFLTGSDRIPILGM

SEQ ID NO: 3 - protein, 23 aa, Homo sapiens
GKVLELFQPSELRAMMVGNSNYN

SEQ ID NO: 4 - protein, 15 aa, Homo sapiens
LLFLTGSDRIPIYGM

SEQ ID NO: 5 - skipped

SEQ ID NO: 6 - protein, 15 aa, Homo sapiens
LVFLTGTDRLQMKDL

SEQ ID NO: 7 - protein, 23 aa, Homo sapiens
KEILRHFYPEELMTAIIGNTDYD

SEQ ID NO: 8 - protein, 15 aa, Homo sapiens
LFFLTGRDRLHARGI

SEQ ID NO: 9 - protein, 23 aa, Homo sapiens
LEWLRYFDEKELELMLCGMQEID

SEQ ID NO: 10 - protein, 15 aa, Homo sapiens
LQFVTGTCRLPVGGF

SEQ ID NO: 11 - protein, 23 aa, Homo sapiens
LQWLQYFDEKELEVMLCGMQEVD

SEQ ID NO: 12 - protein, 15 aa, Homo sapiens
LQFVTGTCRLPLGGF

SEQ ID NO: 13 - protein, 23 aa, Homo sapiens
QQYLQYFDAKELEVLLCGMQEID

SEQ ID NO: 14 - protein, 15 aa, Homo sapiens
LQFVTGTCRLPVGGF

SEQ ID NO: 15 - protein, 23 aa, Homo sapiens
IDLIKIFDENELELLMCGLGDVD

SEQ ID NO: 16 - protein, 15 aa, Homo sapiens
LQFVTGTSRVPMNGF

SEQ ID NO: 17 - protein, 23 aa, Homo sapiens
QDLIKIFDENELELLMCGLGDVD

SEQ ID NO: 18 - protein, 15 aa, Homo sapiens
LQFVTGTSRVPMNGF

SEQ ID NO: 19 - protein, 23 aa, Homo sapiens
QHLLKPFDQKELELIIGGLDKID

SEQ ID NO: 20 - protein, 15 aa, Homo sapiens
LQFVTGSTRVPLQGF

SEQ ID NO: 21 - protein, 23 aa, Homo sapiens
QHLLKTFDEKELELIICGLGKID

SEQ ID NO: 22 - protein, 15 aa, Homo sapiens
LQFVTGSSRVPLQGF

SEQ ID NO: 23 - protein, 23 aa, Homo sapiens
ARLVSVFDARELELVIAGTAEID

SEQ ID NO: 24 - protein, 15 aa, Homo sapiens
LQFVTGTSSIPYEGF

SEQ ID NO: 25 - protein, 23 aa, Homo sapiens
SRLVSVFDARELELVIAGTAEID

SEQ ID NO: 26 - protein, 15 aa, Homo sapiens
LQFVTGTSSVPYEGF

SEQ ID NO: 27 - protein, 23 aa, Homo sapiens
KRLISIFTEQELELLISGLPTID

SEQ ID NO: 28 - protein, 15 aa, Homo sapiens
LQFVTGTSKVPLQGF

SEQ ID NO: 29 - protein, 23 aa, Homo sapiens
PSLIQLFDEYELELLLSGMPEID

SEQ ID NO: 30 - protein, 15 aa, Homo sapiens
LQFVTGSSRVPHGGF

SEQ ID NO: 31 - protein, 23 aa, Homo sapiens
SPLKYLFRPEEIELLICGSRNLD

SEQ ID NO: 32 - protein, 15 aa, Homo sapiens
LQFTTGTDRAPVGGL

SEQ ID NO: 33 - protein, 23 aa, Homo sapiens
SNALMLLRPEEVEILVCGSPDLD

SEQ ID NO: 34 - protein, 15 aa, Homo sapiens
LHFTTGSDRVPVGGM

SEQ ID NO: 35 - protein, 23 aa, Homo sapiens
ENLLAIFDENELELLMCGTGDIS

SEQ ID NO: 36 - protein, 15 aa, Homo sapiens
LQFTTGSSQLPPGGF

SEQ ID NO: 37 - protein, 23 aa, Homo sapiens
VPLLSLLTAKQLEQMVCGMPEIS

SEQ ID NO: 38 - protein, 15 aa, Homo sapiens
MRFVSGRSRLPANTA

SEQ ID NO: 39 - protein, 23 aa, Homo sapiens
VPLLSLFTGYELETMVCGSPDIP

SEQ ID NO: 40 - protein, 14 aa, Homo sapiens
LRFVWGRTRLPRTI

SEQ ID NO: 41 - protein, 23 aa, Homo sapiens
QAVLDLLTWQELEKKVCGDPEVT

SEQ ID NO: 42 - protein, 15 aa, Homo sapiens
LRFVTGRSRLPARIY

SEQ ID NO: 43 - protein, 23 aa, Homo sapiens
LEWLRMFDQQEIQVLISGAQVPI

SEQ ID NO: 44 - protein, 15 aa, Homo sapiens
LKFVTSCSRPPLLGF

SEQ ID NO: 45 - protein, 23 aa, Homo sapiens
PEWIRMFSTPELQRLISGDNAEI

SEQ ID NO: 46 - protein, 15 aa, Homo sapiens
LKFVTSCSRPPLLGF

SEQ ID NO: 47 - protein, 23 aa, Homo sapiens
MEKLSSFSHEEVQMILCGNQSPS

SEQ ID NO: 48 - protein, 15 aa, Homo sapiens
LQFTTGCSTLPPGGL

SEQ ID NO: 49 - protein, 23 aa, Homo sapiens
KNSLEDLTAEDFRLLVNGCGEVN

SEQ ID NO: 50 - protein, 15 aa, Homo sapiens
VYFWTSSPSLPASEE

SEQ ID NO: 51 - protein, 23 aa, Homo sapiens
LSHLQYFYPEELDQLLCGSKADT

SEQ ID NO: 52 - protein, 15 aa, Homo sapiens
LQFVTGSPRLPVGGF

(19) United States
(12) Patent Application Publication
(10) Pub. No.: US 2025/0266123 A1
(43) Pub. Date: Aug. 21, 2025

(54) IN SILICO METHOD OF IDENTIFYING ALLOSTERIC HECT E3-LIGASE INHIBITORS

(71) Applicant: Ramot at Tel-Aviv University Ltd., Tel Aviv (IL)

(72) Inventor: Gali PRAG, Tel Aviv (IL)

(21) Appl. No.: 19/006,833

(22) Filed: Dec. 31, 2024

Related U.S. Application Data
(60) Provisional application No. 63/555,170, filed on Feb. 19, 2024.

(51) Int. Cl. G16B 15/30; C12N 9/10; G16B 35/20

(52) U.S. Cl. CPC G16B 15/30; C12N 9/104; G16B 35/20; C12Y 203/02

(57) ABSTRACT

The disclosure relates to methods for predicting allosteric inhibition of homologous to E6AP C-terminus (HECT) E3-ligases. The methods include in silico approaches for identifying small molecule allosteric inhibitors by threading amino acid sequences of target HECT ligases onto a template protein structure in its inhibited state, such as SMURF1, which comprises a cryptic allosteric cavity remote from the catalytic site and a glycine-hinge domain. The disclosure also enables screening and prediction of small molecule candidates capable of inducing allosteric changes, including elongation of the αH10 helix and shortening of the glycine-hinge domain, thereby restraining motion, essential for catalysis. The disclosed methods additionally provide validation of the predictions through structural and biochemical assays.

### STATEMENT OF PRIORITY

This application claims priority to U.S. Provisional App. No. 63/555,170 for A Silico Method of Identifying Allosteric HECT E3-Ligase Inhibitors.

### INCORPORATION BY REFERENCE OF SEQUENCE LISTING XML

A Sequence Listing is provided herewith as a Sequence Listing XML, “FRAMO-P045-WO.xml”, created on Dec. 30, 2024, and having a size of 45,565 bytes. The contents of the Sequence Listing XML are incorporated by reference herein in their entirety.

### BACKGROUND

Ubiquitylation tightly regulates protein levels, activity, and localization in response to physiological and pathological stimuli. Dysregulation of this process is a critical contributor to human disease. The human genome encodes ˜650 E3-ligases, each ubiquitylating a limited number of targets, thereby providing specificity within the system. Unlike E1 ubiquitin (Ub) activating enzymes and deubiquitylases, which present confined active site pockets for inhibition, the active sites of E2-conjugating enzymes and most E3-ligases protrude from the protein surface. This absence of an active-site pocket limits small molecule binding and impedes computational approaches to identifying inhibitors (FIG. 1A).

Artificial intelligence (AI), machine learning (ML) and other in silico methodologies have significantly advanced the ability to filter and design novel small molecule protein modulators. These algorithms rely on available protein structures. However, protein structures are inherently dynamic, and current methodologies often provide snapshots of only the most stable conformations, overlooking transient pockets and cavities that could be exploited for drug design. Consequently, potential dynamic pockets and cavities may go unobserved. These challenges have limited the development of specific E3-ligase inhibitors. E3-ligases hold promise as drug targets; however, as of now, there are no E3 inhibitors currently in clinical use.

The Homologous to E6AP C-terminus (HECT) domain group of E3-ligases has direct links to human disease. Despite their therapeutic potential, there are currently no approved selective HECT inhibitors, making them an attractive starting point for the discovery of novel druggable features. Among these, the HECT family member SMURF1, is implicated in multiple diseases for which new therapies are urgently needed, including cancer, bone, pulmonary, and central nervous system disorders. At the cellular level, SMURF1 acts as a negative regulator of bone morphogenetic protein (BMP) signaling, which modulates cell migration, proliferation, and apoptosis. The structure of SMURF1 is typical of a Nedd4 subfamily of HECTs, with an N-terminal C2 domain for cellular membrane localization, WW domains for target recognition, and a catalytic HECT domain that binds E2 and mediates target ubiquitylation through a surface-protruding catalytic cysteine. Ubiquitin transfer requires a motion facilitated by a flexible hinge that connects the HECT domain N- and C-lobes (FIG. 1B, and FIG. 7). Despite extensive research, little is known about the features that facilitate or inhibit this crucial motion.

There is a compelling rationale for SMURF1 as a therapeutic target for the treatment of pulmonary arterial hypertension (PAH). Heterozygous mutations of bone morphogenic protein receptor 2 (BMPR2) are present in 15-40% of idiopathic cases of PAH and reduced BMPR2 protein expression is observed in nongenetic forms of the disease and animal models. Most BMPR2 mutations cause haploinsufficiency, resulting in reduced BMP signaling. This reduction drives the proliferation and apoptosis resistance of pulmonary artery endothelial and smooth muscle cells, which contribute to increased pulmonary vascular resistance, right heart failure, and death. Data demonstrate that enhancing BMP signaling provides therapeutic benefits in experimental PAH and that restoring balance of the BMP/transforming growth factor beta (TGFβ) pathways provides clinical benefit.

Expression of SMURF1 is increased in patients with PAH, and global deletion of SMURF1 provides allele-dependent protection in a mouse model of PAH. Based on the established Mendelian association of mutations within the BMP pathway with disease, the central role of BMP and transforming growth factor beta (TGFβ) signaling in PAH development and the negative regulatory role of SMURF1 on BMP signaling, the present disclosure demonstrates that inhibition of SMURF1 can augment BMP signaling and thereby offer a treatment method for PAH.

To circumvent the limitations resulted by the lack of active site pocket that allows in-silico/ML based screen for inhibitors, a large unbiased high-throughput screen (HTS) screen of approximately 1.1 million compounds was performed resulting in the identification of inhibitors of SMURF1 inhibitors. In a large unbiased physical screen SMURF1 inhibitors were identified. Interestingly, although SMURF1 and SMURF2 are highly similar (with 86% protein sequence identity). Unexpectedly, several potent (IC50 in nM-μM range) inhibitors that demonstrate selectivity for SMURF1 over SMURF2 were identified (FIGS. 8D and 9).

According to the present disclosure, downstream x-ray crystallography studies revealed that several identified compounds bind an allosteric binding site located remote from the catalytic cysteine of the ligase and induce an extension of the α10-helix over a conserved glycine-hinge to restrain a required motion between the C- and N-lobes. The present disclosure describes the mechanism of SMURF1 inhibition in atomic resolution and demonstrates how inhibition of SMURF1 prevents direct BMPR2 ubiquitylation, normalizes BMP signaling, restores pulmonary vascular cell homeostasis, and reverses pathology in established, experimental PAH. The present disclosure utilized a thorough computational screen to identify inhibitors of HECT E3-ligase which has known roles in the pathology of cervical cancer, and Angelman and autism syndromes.

### SUMMARY

Targeting ubiquitin E3-ligases is therapeutically attractive; however, the absence of an active-site pocket impedes computational approaches for identifying inhibitors. According to the present disclosure, in a large, unbiased biochemical screen, inhibitors were discovered which bind to an allosteric binding site distinct from the catalytic cysteine of the HECT E3-ligase, SMURF1. In some cases, the allosteric binding site or cavity can be a cryptic cavity. Structural and biochemical analyses, and engineered escape mutants, revealed that these inhibitors restrict an essential catalytic motion by extending an α-helix over a conserved-glycine hinge. SMURF1 levels are increased in pulmonary arterial hypertension (PAH), a disease caused by mutation of bone morphogenetic protein receptor-2 (BMPR2). The present disclosure demonstrates that SMURF1 inhibition prevents BMPR2 ubiquitylation, normalizing BMP signaling, restoring pulmonary vascular cell homeostasis, and reversing pathology in established experimental PAH. Leveraging this deep mechanistic understanding, an in-silico machine learning-based screen was employed to identify inhibitors of the prototypic HECT E6AP and confirmed glycine hinge-dependent allosteric activity in vitro. Inhibiting HECTs and other glycine-hinge proteins opens a new druggable space.

More than 600 ubiquitin E3-ligases tightly regulate the entire proteome in eukaryotes. Increased E3 activity is involved in many diseases; however, there are currently no inhibitors in clinical use. The active site of HECT is protruded and, thus, it is difficult to identify a small molecule that inhibits its activity. An unbiased screen according to the present disclosure resulted in discovery of a mechanism for inhibiting HECT ligases. The disclosers deciphered the molecular mechanism of this inhibitor and demonstrated its allosteric activity by shortening and restricting a glycine-hinge motion essential for the enzyme catalytic activity. Leveraging this deep mechanistic understanding enabled the presently disclosed novel in silico procedure for identifying inhibitors of other HECT E3s. Allosteric restriction of the motion of essential glycine-hinge domains of proteins opens a new druggable space.

Inhibition of ubiquitin E3-ligases is therapeutically attractive; however, the absence of an active-site pocket impedes computational approaches to inhibitor identification. In a large unbiased physical screen, inhibitors were discovered that bind an allosteric cavity distinct from the protruding SMURF1 catalytic cysteine. Structural and biochemical analyses, and engineered escape mutants, revealed that the inhibitors restrict an essential catalytic motion by extending an α-helix over a conserved glycine-hinge. SMURF1 is overexpressed in patients with pulmonary arterial hypertension (PAH), a disease caused by mutation of bone morphogenetic protein receptor-2 (BMPR2). It is demonstrated that SMURF1 inhibition prevents direct BMPR2 ubiquitylation, normalizes BMP-signaling, restores pulmonary vascular cell homeostasis and reverses pathology in established, experimental PAH. The present disclosure relates to an in-silico screen that can identify small molecule inhibitors such as HECT E3-ligase inhibitors that inhibit enzymatic activity by binding an allosteric site and inducing allosteric enzymatic inhibition. Inhibition of HECT E3s and other glycine-hinge proteins opens a new druggable space.

Therefore, based on the foregoing and continuing description, the subject invention in its various embodiments may comprise one or more of the following features in any non-mutually-exclusive combination:

An in silico method of identifying a small molecule allosteric inhibitor of a target protein family, comprising identifying a template protein member of the target protein family, the template protein member comprising a hinge domain, an allosteric binding site, and a catalytic site, the allosteric binding site different from the catalytic site, wherein binding of a small molecule allosteric inhibitor induces an allosteric change in the template protein member thereby inhibiting catalytic activity;

An in silico method of identifying a small molecule allosteric inhibitor of a target protein family, comprising in silico modeling of the allosteric binding site of an inhibited structure of the template protein member by threading corresponding amino acid sequences of members of the target protein family onto the inhibited structure of the template protein member to create a model target protein;

An in silico method of identifying a small molecule allosteric inhibitor of a target protein family, comprising in silico screening of a group of small molecules to identify small molecule allosteric inhibitor candidates predicted to bind to the allosteric binding site of the model target protein;

An in silico method of identifying a small molecule allosteric inhibitor of a target protein family, comprising screening the small molecule allosteric inhibitor candidates to identify at least one small molecule allosteric inhibitor of the target protein family that induces an allosteric change in the target protein family upon binding to the allosteric binding site.

An in silico method of identifying a small molecule allosteric inhibitor of a target protein family, wherein the hinge domain is a glycine-hinge domain.

An in silico method of identifying a small molecule allosteric inhibitor of a target protein family, wherein the template protein member comprises an elongated αH10 traversing a conserved glycine residue, an N-lobe, a C-lobe, and a shortened hinge between the N-lobe and the C-lobe.

An in silico method of identifying a small molecule allosteric inhibitor of a target protein family, wherein the template protein member comprises a lock mechanism in an inhibited state enabled by an amino acid (AA) pair, the AA pair comprising a first AA and a second AA, the first AA disposed at the end of an elongated αH10, and the second AA disposed on the C-lobe, said AA pair creating a non-covalent bond between them.

An in silico method of identifying a small molecule allosteric inhibitor of a target protein family, wherein the AA pair comprises D636 and R686.

An in silico method of identifying a small molecule allosteric inhibitor of a target protein family, wherein the non-covalent bond is formed between an αH10 D636 and C-lobe R686 of the template protein member;

An in silico method of identifying a small molecule allosteric inhibitor of a target protein family, wherein R686 is further stabilized by N507 of the template protein member, the N507 allosterically shifted toward R686;

An in silico method of identifying a small molecule allosteric inhibitor of a target protein family, wherein the non-covalent bond is a pi bond or an electrostatic bond.

The method of claim 1, An in silico method of identifying a small molecule allosteric inhibitor of a target protein family, wherein the template protein member is a homologous to E6AP C-terminus (HECT) E3-ligase.

An in silico method of identifying a small molecule allosteric inhibitor of a target protein family, wherein the template protein member comprises a lock mechanism in an inhibited state;

An in silico method of identifying a small molecule allosteric inhibitor of a target protein family, wherein the template protein member is selected from a closed group of HECT E3-ligase enzymes selected from the group consisting of HERC4, HERC3, HERC5, HERC6, WWP2, WWP1, ITCH, NEDD4-2, NEDD4-1, SMURF1, SMURF2, HECW2, HECW1, HUWE1, HACE1, UBE3A, HECD2, AREL1, HERC1, HERC2, HECTD3, UBE3C, UBE3B, HECTD1, UBR5, and TRIP12.

An in silico method of identifying a small molecule allosteric inhibitor of a target protein family, wherein the small molecule allosteric inhibitor induces allosteric changes in the template protein member comprising elongation of an αH10 of the HECT E3-ligase over a glycine-hinge thereby restraining motion between C-lobes and N-lobes of the template protein member thereby inhibiting the template protein member.

An in silico method of identifying a small molecule allosteric inhibitor of a target protein family, wherein the template protein member is SMURF1.

An in silico method of identifying a small molecule allosteric inhibitor of a target protein family, wherein the allosteric binding site is a cryptic cavity.

An in silico method for predicting whether a HECT E3-ligase will undergo an allosteric change in response to binding a small molecule allosteric inhibitor, comprising Identifying a template HECT E3-ligase, the template HECT E3-ligase comprising a hinge domain, an allosteric binding site different from a catalytic site, wherein binding of a small molecule allosteric inhibitor induces an allosteric change in the template HECT E3-ligase thereby inhibiting catalytic activity;

An in silico method for predicting whether a HECT E3-ligase will undergo an allosteric change in response to binding a small molecule allosteric inhibitor, comprising Threading amino acid sequences of target HECT E3-ligases onto the inhibited structure of the template HECT E3-ligase to identify target HECT E3-ligases having homologous structure and amino acid sequence to the template HECT E3-ligase when inhibited by the small molecule allosteric inhibitor;

An in silico method for predicting whether a HECT E3-ligase will undergo an allosteric change in response to binding a small molecule allosteric inhibitor, comprising Identifying target HECT E3-ligases predicted to undergo allosteric changes upon small molecule allosteric inhibitor binding to the allosteric binding site.

An in silico method for predicting whether a HECT E3-ligase will undergo an allosteric change in response to binding a small molecule allosteric inhibitor, wherein the hinge domain comprises a glycine-hinge domain.

An in silico method for predicting whether a HECT E3-ligase will undergo an allosteric change in response to binding a small molecule allosteric inhibitor, wherein the target HECT E3-ligase is identified by having an elongated αH10 traversing a conserved glycine residue, an N-lobe, a C-lobe, and a shortened hinge between the N-lobe and the C-lobe when bound by the small molecule allosteric inhibitor.

An in silico method for predicting whether a HECT E3-ligase will undergo an allosteric change in response to binding a small molecule allosteric inhibitor, wherein the template HECT E3-ligase comprises a lock mechanism in an inhibited state enabled by an amino acid (AA) pair, the AA pair comprising a first AA and a second AA, the first AA disposed at the end of an elongated αH10, and the second AA disposed on the C-lobe, said AA pair creating a non-covalent bond between them.

An in silico method for predicting whether a HECT E3-ligase will undergo an allosteric change in response to binding a small molecule allosteric inhibitor, wherein the AA pair comprises D636 and R686.

An in silico method for predicting whether a HECT E3-ligase will undergo an allosteric change in response to binding a small molecule allosteric inhibitor, wherein the non-covalent bond is a pi bond or an electrostatic bond.

An in silico method for predicting whether a HECT E3-ligase will undergo an allosteric change in response to binding a small molecule allosteric inhibitor, wherein the target HECT E3-ligases are identified by having a non-covalent bond between αH10 D636 and C-lobe R686 when bound by the small molecule allosteric inhibitor;

An in silico method for predicting whether a HECT E3-ligase will undergo an allosteric change in response to binding a small molecule allosteric inhibitor, wherein R686 is further stabilized by N507 of the HECT E3-ligase, the N507 allosterically shifted toward R686 when bound by the small molecule allosteric inhibitor;

An in silico method for predicting whether a HECT E3-ligase will undergo an allosteric change in response to binding a small molecule allosteric inhibitor, wherein the target HECT E3-ligase is identified when bound by the small molecule allosteric inhibitor by allosteric changes as compared to when unbound by the small molecule allosteric inhibitor, the allosteric changes comprising elongation of an αH10 and shortening of the hinge domain when bound by the small molecule allosteric inhibitor;

An in silico method for predicting whether a HECT E3-ligase will undergo an allosteric change in response to binding a small molecule allosteric inhibitor, wherein the allosteric binding site is a cryptic cavity.

An in silico method for predicting whether a HECT E3-ligase will undergo an allosteric change in response to binding a small molecule allosteric inhibitor, wherein the template HECT E3-ligase comprises a lock mechanism in an inhibited state.

An in silico method for predicting whether a HECT E3-ligase will undergo an allosteric change in response to binding a small molecule allosteric inhibitor, wherein the template HECT E3-ligase is selected from a closed group of HECT E3-ligase enzymes selected from the group consisting of HERC4, HERC3, HERC5, HERC6, WWP2, WWP1, ITCH, NEDD4-2, NEDD4-1, SMURF1, SMURF2, HECW2, HECW1, HUWE1, HACE1, UBE3A, HECD2, AREL1, HERC1, HERC2, HECTD3, UBE3C, UBE3B, HECTD1, UBR5, and TRIP12.

An in silico method for predicting whether a HECT E3-ligase will undergo an allosteric change in response to binding a small molecule allosteric inhibitor, wherein the template HECT E3-ligase is SMURF1.

The in silico procedure can also include the following steps:

Identify potential candidate enzyme (HECT) for allosteric inhibition by comparing the sequence with the one of SMURF1. The enzyme may contain a) A glycine that stems the hinge connecting the N- and the C-lobes; b) two additional amino acids that may form a non-covalent bond that locks the allosterically elongated α-helix-10 of the N-lobe.

Modeling of the structure of the enzyme in the allosterically inhibited stage, which contains a) Elongated α-helix-10; b) Locking non-covalent bond; c) A new cavity for allosteric inhibition.

In-silico screen for small molecules predicted to stabilize the inhibited structure.

The activity of potential inhibitor candidates derived from the in-silico screen can then be assessed by various biochemical and biophysical assays.

### DESCRIPTION OF THE FIGURES

FIG. 1. SMURF1 and SMURF 2 HECT domain: inhibitor complex. (A) The catalytic cysteine of HECT E3-ligases SMURF1, E6AP, and NEDD4 is located on the external surface of the protein, in contrast to the active-site pocket location in USP7. (B) Superposition of HECT structures in two extreme rotation angles of the C-lobe. NEDD4L (blue), the catalytic cysteine is in close proximity to the E2 and ubiquitin (not shown); Rsp5 (cyan), the catalytic cysteine is facing the target (Sna3; magenta) and the C-terminus of ubiquitin (not shown). (C) Structure of inhibitor-bound SMURF1 (Cpd-8). A slice in the N-lobe reveals the allosteric binding site to be a cryptic cavity. Electrostatic surface potential was calculated with Adaptive Poisson-Boltzmann Solver with the indicated +/−KT/e. (D) Logo sequence shows the residue conservation demonstrating the invariant G634 (FIG. 11). Superposition of 28 HECT structures including inhibitor-bound SMURF1. The conserved glycine (blue spheres) is aligned at the stem of the hinge. The α-helix-10 (αH10) of SMURF1 (magenta) is elongated over the conserved glycine (G634) that is relocated within the αH10. (E) Schematic of the structural changes of αH10 and the altered length/flexibility of the hinge due to inhibitor binding. (F) Structural comparison of SMURF1 and SMURF2 with and without the inhibitor.

FIG. 2. Allosteric inhibition of SMURF1. (A) Illustration of the E. coli split-CAT system showing target ubiquitylation resulting in CAT stabilization and selective growth. (B) Replacement of the SMURF1 conserved glycine with proline (G634P), an amino acid with a covalently linked side chain that limits hinge flexibility, and a deletion that reduced hinge length (Δ637KID) both reduce ubiquitylation. (C) Cpd-8 (blue) reduces ubiquitylation compared to vehicle (black). (D) SMURF1 escape mutant-1 (insertion of GGLD downstream to D636) shows resistance activity to the inhibitor. (E) SMURF1 escape mutant-2 (replacing indicated residues with SMURF2 residues) shows significant resistance to the inhibitor. (F) Relative inhibition of the escape mutants. (G) SMURF1 G636 forms a non-covalent bond that stabilizes the elongated αH10. Mutation of each of the three residues (D636G, R686A, and N507A) results in escape from inhibition. (H) Escape mutant-3 (D636G mutation replaces the amino acid that stabilizes the elongated αH10 with one that does not form a non-covalent bond) shows significant resistance to the inhibitor. (I) Susceptibility mutant (G630D, replacing indicated residues of SMURF2 with SMURF1; SMURF1lation) shows the sensitivity of mutant SMURF2 to the inhibitor. All data: n=4 replicates; presented as mean±SD.

FIG. 3. SMURF1 expression in PAH. (A) Schematic representation of canonical BMP signaling resulting in SMAD1/5/8 phosphorylation, in nuclear translocation of SMAD4 and ID1 expression and its negative regulation by SMURF1-mediated ubiquitylation and degradation of BMPR2 and SMAD1/5/8. (B) In HEK293 cells stably transfected with GFP-tagged SMURF1, BMP4 stimulation results in a decrease in GFP signal, and siRNA knockdown of ACVRL1, BMPR2, ENG, or SMAD9, BMP4 stimulation results in increased GFP signal. n=3 separate experiments; presented as mean±SEM. *p<0.05, **p<0.01, relative to untreated, unpaired t-test. (C) Expression of SMURF1 is increased in PASMC from patients with idiopathic and heritable PAH when compared to PASMC from patients without PAH. n=6 PAH donor lines and n=9 control lines; presented as mean±SEM. **p<0.01, unpaired t-test. (D) SMURF1 expression in the pulmonary artery intima and media in patients with PAH. SMURF1 (purple) protein co-localization with an endothelial (vWF: yellow) or smooth muscle marker (αSMA: yellow) is indicated by a red/brown color shift. Representative images obtained from 19 control and 33 patients with PAH. Scale bar=60 μm, arrows indicate areas of co-localization.

FIG. 4. SMURF1 inhibitors restore BMP signaling and restore pulmonary vascular cells homeostasis. (A) Scheme of SMURF1-BMPR2 and SMAD1 split-CAT based E. coli selection system, showing target ubiquitylation resulting in CAT stabilization and selective growth. (B-C) Hyperactive K381R (black) increases and catalytically inactive C725A (red) reduces SMURF1-dependent direct target ubiquitylation of SMAD1 (B) and BMPR2 (C) (inset represents area-under-the-curve of relative growth; n=3). (D) Representative Western blot demonstrating stabilization of overexpressed SMAD1 in HEK cells in the presence of SMURF1 inhibitor. Mutations that reduce the flexibility (G634P) and length (Δ637KID) of the hinge result in reduced SMURF1 activity. Mutations that preserve the flexibility (G633C, D636G (GGLD)) and length (637GGLDINS) of the SMURF1 glycine hinge escape the effect of the inhibitor. (E) Representative Western blot demonstrating stabilization of overexpressed BMPR2 and BMPR2-myc in HEK cells in the presence of SMURF1 inhibitor. (F) Immunoblotting of BMPR2, SMURF1, SMAD1/5/8, phosphorylated SMAD1/5/8, ID1, and GAPDH in PASMC cultured without or with BMP4 and SMURF1 inhibitor (Cpd-6) (n=9 separate experiments across cells from 3 PAH donor lungs, mean±SEM). (G-H) Quantification of apoptosis in PAECs. (G) Representative time curve (n=5 technical replicates, mean±SD) and (H) group data at 300 min with Cpd-6 or vehicle (n=6 separate donors, mean±SEM). (I-J) Quantification of proliferation: (I) Representative time course (n=5 technical replicates, mean±SD) in PAEC with cell confluence mask for each dose of Cpd-6 or vehicle (orange) and (J) group data (n=3 separate PAEC donors; mean±SEM). (K) Migration of PASMCs with Cpd-6 or vehicle measured via disc closure assay (n=2-3 separate donors). (L) Representative time course plots showing proliferation of PASMC from an idiopathic PAH patient with Cpd-6 or vehicle with cell confluence mask for each dose (orange) (n=5 technical replicates per concentration from one idiopathic PAH donor line, mean±SD). (M) Group proliferation data at 72 hours in PASMCs from patients with idiopathic or hereditary PAH (n=4-5 separate donor lines, mean±SEM). *p<0.05, **p<0.01, 1-way ANOVA with Dunnett's correction, comparisons as indicated.

FIG. 5. Inhibition of SMURF1 treats established experimental PAH. (A) Experimental timeline for the Su-5416 hypoxia study. (B-E) Right ventricular systolic pressure (RVSP) (B: SMURF1 inhibition; C: standard of care and experimental PAH therapies) and (D-E) small vessel pulmonary vascular muscularization are increased with disease and reduced by inhibition of SMURF1 using a small molecule inhibitor (n=5-11). Histology panel (D): bar=50 μm. (F) Experimental timeline for the monocrotaline study. (G-J) RVSP (G: SMURF1 inhibition; H: standard of care and experimental PAH therapies) and (I-J) small vessel pulmonary vascular muscularization are increased with disease and reduced by inhibition of SMURF1 using a small molecule inhibitor (n=5-10). Histology panel (I): bar=50 μm. All bar graphs presented as mean±SEM, *p<0.05, **p<0.01, ***p<0.001, ****p<0.0001, 1-way ANOVA with Dunnett's correction, comparisons as indicated.

FIG. 6. Structure-based identification of E6AP inhibitors. (A) Schematic representation of the approach to hit identification. Remodeled HECT domain is formed by tethering on the inhibited SMURF1 structure and allosteric binding site used for a ML screen, followed by an E. coli-based validation. (B) Altered E6AP activity is associated with human disease. (C) Structure of the HECT glycine hinge and location of self-ubiquitylated lysine. (D) Glycine 738 stems the hinge between the N- and C-lobes in E6AP with the lysine self-ubiquitylation target located at 466 (which corresponds to NEDD4 G779 and K525). (E) E6AP-dependent Rpn10 ubiquitylation split-CAT reporter system. (F) Activity of the constitutively active K466R E6AP is reduced by G738E mutation, demonstrating the importance of the glycine hinge. (G) Mass-spectrometry analysis showing self-ubiquitination of E6AP at K466. (H) Hit prioritization heatmap of 32 compounds derived from the ML screen and examined in the E. coli split-CAT system. Percentage inhibition is represented by a color scale from red (maximum inhibition) to blue (minimum inhibition). (I) Inhibition of E6AP with compound i-27 in E. coli split-CAT system and the dose-response curve for E6AP:i-27. (J) Structural rearrangements of the E6AP glycine hinge in the presence of compound i-27. (K) Activity of the E6AP S739L, R740G escape (SMURF2lation) mutant in the presence of compound i-27.

FIG. 7. A: Image showing the structural rearrangements of the C- and N-lobe of SMURF1 around the flexible hinge that are required to transfer ubiquitin from the catalytic cystine of the E2 to the protein target.

FIG. 8. HTS strategy and activity of lead compounds from prioritized chemical series. A. Schematic showing the biochemical TR-FRET assays used for detection of SMURF1 auto-ubiquitylation. B. Cell-based assay: detection of E3 ligase auto-ubiquitylation, assay used for the validation screen and selectivity profiling. C. Process used for triaging of initial hits through to identification of lead series. D. Overview of pharmacological activity for three prioritized chemical hit classes. Plots show dose-response in SMURF1 (blue) and SMURF2 (red) biochemical assays (n=4-11). Tables below indicate IC50 values for respective inhibitors in SMURF1 and SMURF2 biochemical assays and SMURF1 cellular assay (n=2-474).

FIG. 9. Table indicating the selectivity of SMURF1 inhibitors across representative structurally related HECT family members. (n=4-20)

FIG. 10. Structural details of inhibitor binding to SMURF1. A. Surface representation of SMURF1 showing that the inhibitor is barely exposed. B. Detailed structural insight into the SMURF1: Inhibitor complex with zooming into the binding cavity from three different angles. C. 2D representation of the inhibitor binding site. D. mFo-DFc simulated annealing electron density omit-map showing the α10 and the hinge region without (left) and with (right) the inhibitor. The map was calculated by omitting the entire model of the α10, the hinge and the inhibitor using simulated annealing sigma A analysis; contoured at 2.75 Å (SMURF1 apo) and 2.05 Å (SMURF1+Cpd 8) at 3 s level.

FIG. 11. Table showing alignment of multiple HECT E3-ligases showing the conserved glycine residue at the hinge connecting the N- and C-lobes. The conserved Gs (glycine 634 of SMURF1; first line) are colored. The sequence of yeast HECT E3-ligase Rsp5 was used as a probe for BLAST and retrieved 5000 HECT sequences from the ‘non-redundant protein sequences (nr)’ at NCBI. The proteins were aligned by fast Fourier transform based multiple alignment MAFFT v7.158b (Jun. 27, 2014 http://mafft.cbrc.jp/alignment/software/MBE 30:772-780 (2013), NAR 30:3059-3066 (2002).

FIG. 12. Comparison of small molecule SMURF1 inhibition and siRNA-mediated SMURF1 knockdown on BMP signaling (BMP response element-ID1 promoter activation) in HEK293 cells. (B-D) Summary of expression proteomics experiments comparing PASMC cells treated under hypoxic conditions +/−BMP4 or +/−SMURF1 inhibitor Cpd-6 (SMURF1i). (B) Experimental workflow. Table: sample conditions for PASMC BMPR2 (C347Y) mutant cells undergoing TMT quantitative proteomics profiling at 24 hr. (C) Log 2 ratios of protein abundances for treated versus control (DMSO) in selected conditions. Data represent two biological replicates (n=2) per treatment condition in a single experiment derived from individual donors. Highlighted proteins correspond to ‘IL-1 beta- and Endothelin-1-induced fibroblast/myofibroblast migration and extracellular matrix production in asthmatic airways’ gene set, which was significantly enriched among dysregulated proteins, only in condition FIG. 12B (ii). Inserts show heatmap for ratios for donor 1 and 2 for proteins highlighted in the plot. (D) Log 2 ratio of known SMURF1 targets RhoA and TGFBR1 detected in proteomic study and chemiluminescence of SMURF1, BMPR2, SMAD1, pSMAD1, and ID1 in samples used for proteomic studies measured by Western (not detected by proteomics). (E) Summary of significantly enriched terms for mutant cells under hypoxic conditions pre-treated with BMP4, followed by treatment with SMURF1 inhibitor or DMSO.

FIG. 13. (A) Screen for modulators of SMURF1 mRNA abundance in PASMCs. Human PASMCs from PAH patients were treated with agents that are associated with PAH, including cytokines, chemokines, growth factors, and small molecule pathway probes, and were cultured under hypoxic conditions to identify modulators of SMURF1 mRNA (n=2 donors). The figure shows normalized Ct values (ΔΔCt method). The BMP agonist FK506 (tacrolimus) was found to reduce SMURF1 expression. Both hypoxic conditions and TGFβ1 treatment increase SMURF1 expression, supporting SMURF1 induction as part of a feedback loop regulating BMP and TGFβ1 signaling. (B) in silico analysis indicates that the SMURF1 promoter contains two binding sites for the hypoxia-regulated transcription factor, Hypoxia Inducible Factor 1 Subunit Alpha (HIF1α). The human SMURF1 promoter was stably transfected into HEK293 cells upstream of a GFP sequence. Administration of TGFβ1 and bafilomycin A1 increased GFP signal.

FIG. 14. Split-CAT-based E. coli system monitoring SMURF1 and SMURF2 activity (growth curves with cumulative growth indicated as inset bars). A—SMURF1 dependent ubiquitylation of Rpn10: Wild-type (blue), K381R mutant (black), and catalytically inactive C725A (red). B—SMURF1 dependent ubiquitylation of the phosphomimetic peptide of SMAD1 (black) with D636G mutation (blue). C—SMURF2 dependent ubiquitylation of Rpn 10 with (black) or without (blue) Cpd-8. D-SMURF2 dependent ubiquitylation of Rpn10 (black) with G630D mutation (pink). N=4 replicates, mean±SD.

FIG. 15. (A) In unbound-SMURF1, I631 of the αH10 (and other nearby residues) occupies the allosteric binding site, which is therefore not available for small molecule inhibitor binding. (B) In the presence of Cpd-8, the αH10 is pushed away, resulting in allosteric elongation. In this altered conformation, an electrostatic bond is formed between D636 in the αH10 and R686 located on the C-lobe.

FIG. 16. Evaluation of structure-based allosteric mechanism of series representative compounds. The SMURF1 inhibition modes of representative compounds from each of the three chemical series (Piperidine, Pyrazolone, and Pyrrole): Cpd-3, Cpd-6, and Cpd-8 were assessed in the Split-CAT assay. The compounds were assessed against hyperactive (K381R mutant)—white bars or an escape mutant (K381R, G633C, D636F)—blue bars. (A) Shows relative inhibition of SMURF1 by each of the compounds using Rpn10 as ubiquitylation target reporter (mean±SE; n=3 replicates). (B-C). As in A, but Rpn10 was replaced with a phosphomimetic peptide of SMAD1 as ubiquitylation target reporter. n=4 replicates, mean±SD.

FIG. 17. Representative histology sections of explanted lungs from patients with and without pulmonary arterial hypertension. Dual staining was performed for vWF (brown) and αSMA (pink), and single staining for SMURF1 (brown). Each set of two images represents a unique patient (total: n=15 control and n=19 PAH patients). Scale bar=60 μm. Plot: quantification of pulmonary vascular muscularization in above histological images (M—full muscularization, NM—no muscularization).

FIG. 18. LigPlot scheme of the E6AP:i27 interaction. Residues that form the cavity in E6AP and interact with the inhibitor i27 are presented. The chemical structure of the inhibitor (i-27): 1-[1-(4-methylphenyl)-imidazol-2-yl]-4-[adamantane-1-carbonyl]piperazine is shown at the center.

FIG. 19. Structures of HECT ligase showing conservation of lysine residues on α-helix #1. Superimposing the structures of Rsp5 (3OLM), NEDD4 (2XBB), and SMURF1 and the AlphaFold model of E6AP/UBE3A HECT domains shows the conservation of lysine (K) residues on α-helix #1, previously demonstrated to undergo self-ubiquitylation that downregulates the ligase activity.

FIG. 20. Sequence alignment of human HECT domains showing the conservation of the critical residues required for allosteric inhibition. Marked with underscore, are sequences that contains the conserved glycine and a pair of residues with potential to form a non-covalent bond which can lock the elongated αH10.

### DETAILED DESCRIPTION

### Background of SMURF1 Associated Pathologies

### SMURF1 is Over-Expressed in Patients With PAH

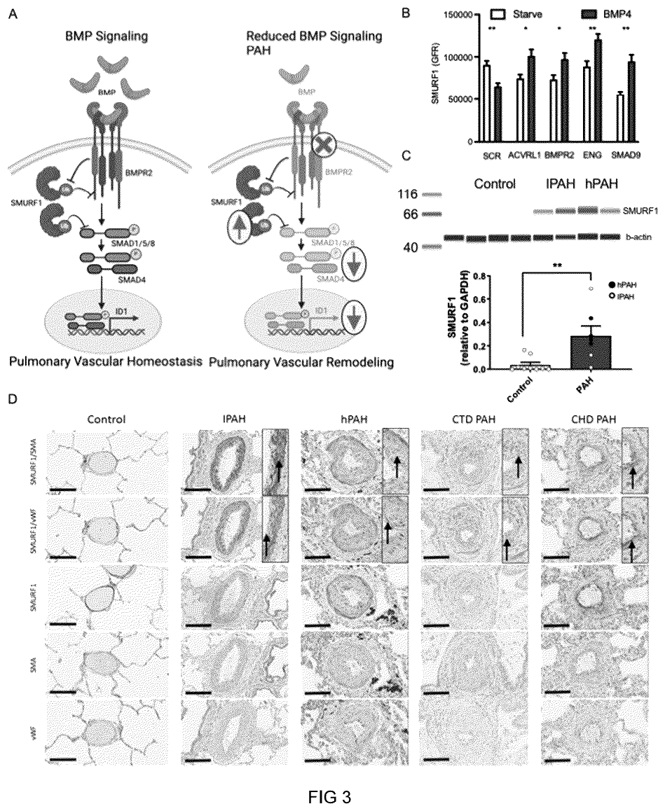

Human genetic evidence implicates reduced BMP signaling as an initiating factor in PAH. Ligand binding to the BMP receptor complex results in release of TRIB3 which negatively regulates SMURF1 autoubiquitylation (FIG. 3A). SMURF1 itself ubiquitylates key mediators of the BMP pathway for degradation, including SMAD. As such, reduced BMP signaling results in increased SMURF1 activity and a further reduction of BMP signaling. Additionally, SMURF1 expression is induced by hypoxic conditions or TGFβ treatment, both key mediators of vascular remodeling implicating SMURF1 as a key mediator of reduced BMP signaling and subsequent vascular remodeling in PAH.

To understand the effect of disease-associated mutations on SMURF1 regulation, HEK293 cells were stably transfected with GFP-tagged SMURF1. Consistent with prior reports, stimulation with BMP4 reduced GFP-SMURF1 (FIG. 3B). However, following siRNA mediated reduction of disease-related TGF-β superfamily receptors ACVRL1, BMPR2 and ENG, GFP-SMURF1 was increased in response to BMP4 (FIG. 3B).

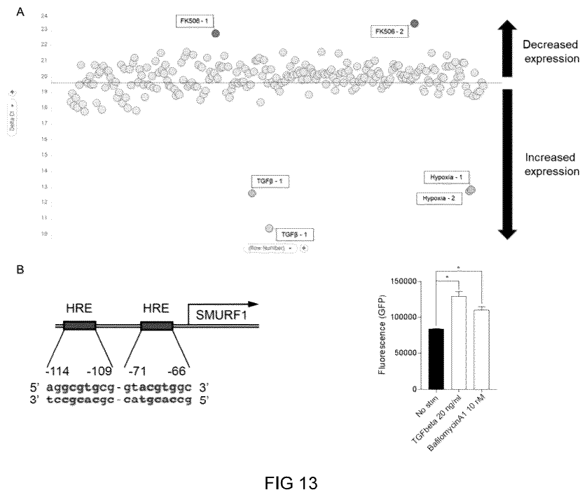

Histologically, PAH is characterized by endothelial and smooth muscle cell remodeling of the small pulmonary arteries in which increased expression of SMURF1 is expected. To investigate the penetrance of this finding, SMURF1 expression levels within the small pulmonary arteries was examined across a large cohort of 33 individual PAH patients. A significant increase in SMURF1 expression in endothelial and smooth muscle cells was observed in all patients compared with samples from 19 patients without PAH. The present disclosure represents the most comprehensive assessment to date of SMURF1 expression in PAH patient lungs (FIGS. 3C and 3D and 13).

### SMURF1 Inhibition Augments BMP Signaling

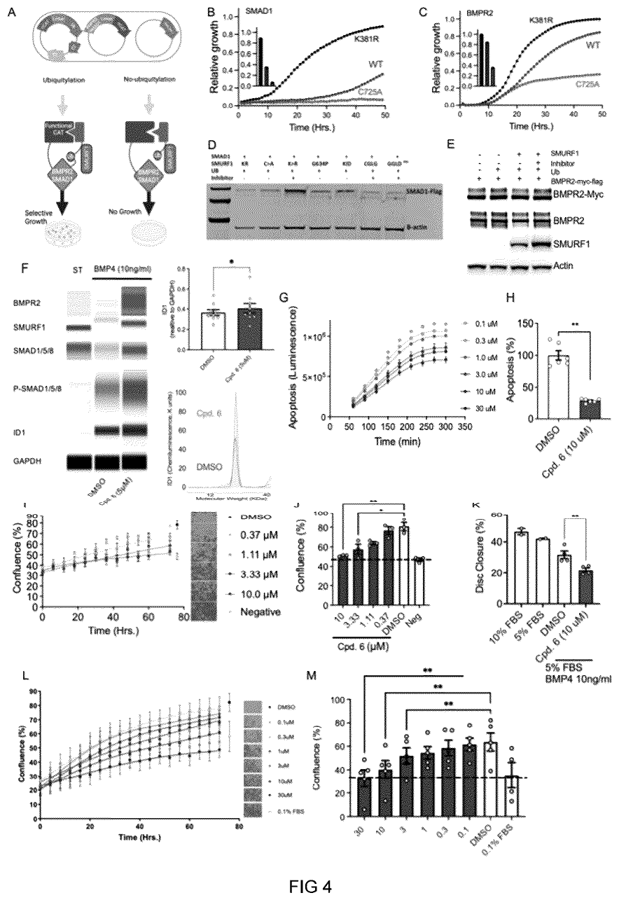

To investigate the role of allosteric SMURF1 inhibition in BMP signaling in disease-relevant cells, pulmonary artery smooth muscle cells from patients with idiopathic and heritable PAH were stimulated with BMP4 and cultured in the presence of a SMURF1 inhibitor or vehicle. BMP4 stimulation resulted in phosphorylation of SMAD1/5/8 and an increase in ID1 expression (FIG. 4A). Compared to vehicle control, allosteric inhibition of SMURF1 increased protein levels of BMPR2, unphosphorylated SMAD1/5/8, phospho-SMAD1/5/8 and ID1 which together, demonstrate that the presently disclosed SMURF1 inhibitors augment BMP signaling in pulmonary vascular cells.

### SMURF1 Directly Ubiquitylates BMPR2

SMAD1 is a direct substrate of SMURF1 however, interactions with SMAD6 or SMAD7 are thought to bridge SMURF1 to the receptor (BMPR2) for ubiquitylation and degradation. To determine if BMPR2 is a direct target of SMURF1, the intracellular domain of BMPR2 was expressed with SMURF1 in a split-CAT-based E. coli selection system (FIG. 4B). Compared to wild-type SMURF1, the hyperactive ΔC2-K381R SMURF1 mutant SMURF1 increased, and the C725A catalytic inactive mutant reduced, ubiquitylation (FIG. 4C). As E. coli lack the accessory proteins to bridge SMURF1 to BMPR2 (SMAD6 and SMAD7), these data demonstrate that BMPR2 is a direct ubiquitylation target of SMURF1.

### SMURF1 Inhibition Reestablishes Pulmonary Vascular Homeostasis

Modulation of BMP signaling, through either genetic or pharmacological manipulation is known to alter key cellular phenotypes related to the pathology of PAH. BMP9 is the predominant BMP ligand that regulates pulmonary vascular endothelial homeostasis and is protective against vascular remodeling associated with PAH. Consistent with the identified increase in BMP signaling in human primary pulmonary artery endothelial cells, it was found that in the presence of a low BMP9 concentration, inhibition of SMURF1 effectively reduced pulmonary artery endothelial cell apoptosis and proliferation (FIGS. 4D-G). Similarly, in the presence of BMP4, the predominant ligand that regulates pulmonary vascular smooth muscle homeostasis, reduced proliferation and migration was observed with SMURF1 inhibition in primary pulmonary artery smooth muscle cells from patients with PAH with and without mutation of BMPR2 suggesting anti-remodeling potential of SMURF1 inhibition (FIGS. 4G-J).

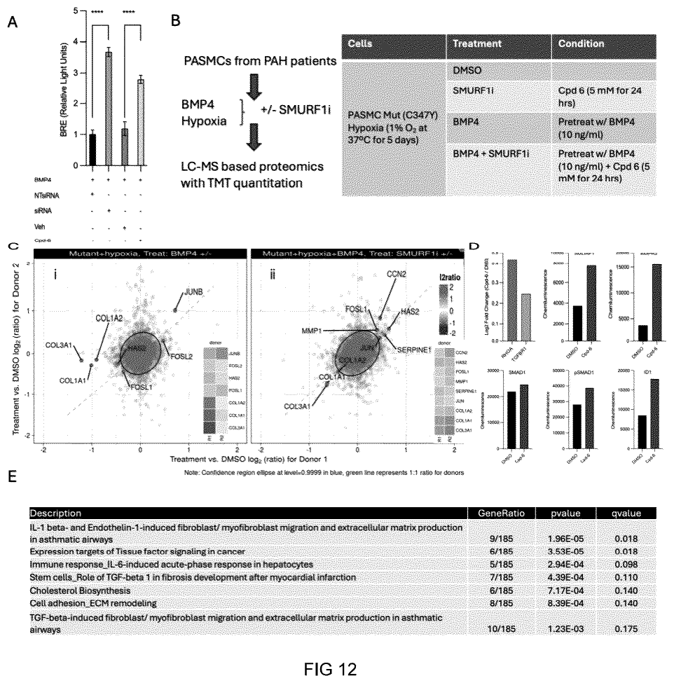

To determine the effects of allosteric SMURF1 inhibitors on the global proteome, pulmonary artery smooth muscle cells (PASMCs) from patients with PAH carrying a disease-causing BMPR2 mutation were pre-treated with BMP4 under hypoxic conditions followed by the SMURF1 inhibitor compared to vehicle for 24 hours and lysates evaluated by quantitative proteomics. Protein set enrichment analysis identified modulation of multiple processes implicated in PAH through SMURF1 inhibition including TGFβ and IL-1β signaling and extracellular matrix (ECM) remodeling and fibrosis (FIG. 12).

### SMURF1 Inhibition Reverses Established, Experimental PAH Pathology

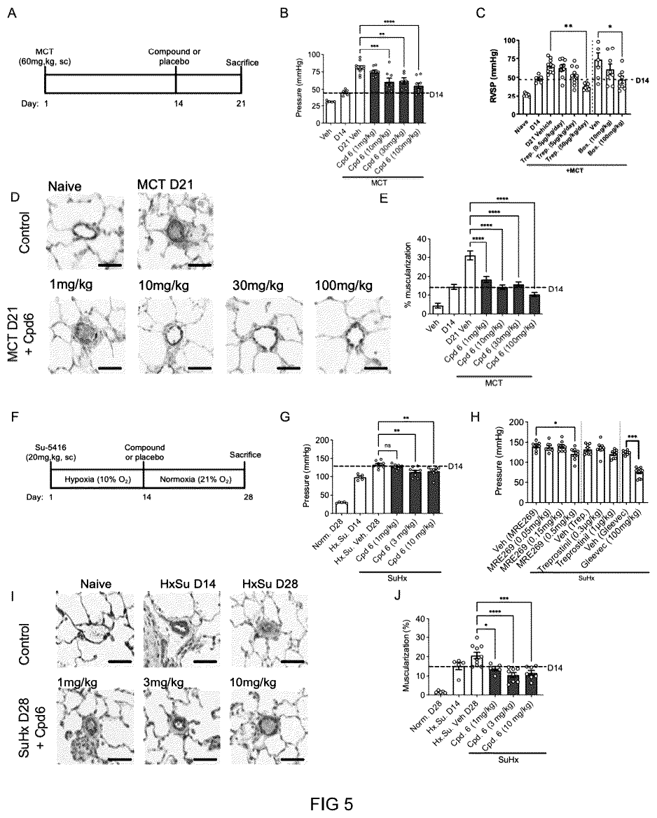

Based on molecular and cellular data, it was hypothesized that administration of an allosteric SMURF1 inhibitor would restore BMP signaling in animal models of PAH in which reduced BMPR2 expression is a central feature. Pulmonary hypertension was induced in Wistar Kyoto rats by administration of monocrotaline (MCT) or by treatment with SU5416 under hypoxic conditions (FIGS. 5A and 5E). Pulmonary vascular phospho-SMAD1/5/8 staining was reduced in animals with PAH and was restored to pre-disease levels by administration of an allosteric inhibitor of SMURF1 (FIGS. 5B and 5F). Twenty-one days after administration of rats with monocrotaline and twenty-eight days after the administration of SU5416 under hypoxic conditions (10% O₂), right ventricular systolic pressure and pulmonary artery remodeling were increased. Consistent with the restoration of pulmonary vascular phospho-SMAD1/5/8 staining observed, allosteric inhibition of SMURF1 improved right ventricular systolic pressure (FIGS. 5C and 5G) and pulmonary artery remodeling (FIGS. 5D and 5H) in a dose dependent manner.

### SMURF1 Glycine-Hinge Function Investigation

### Selective Inhibitors Restrict Crucial Glycine-Hinge Motion Within the HECT Domain

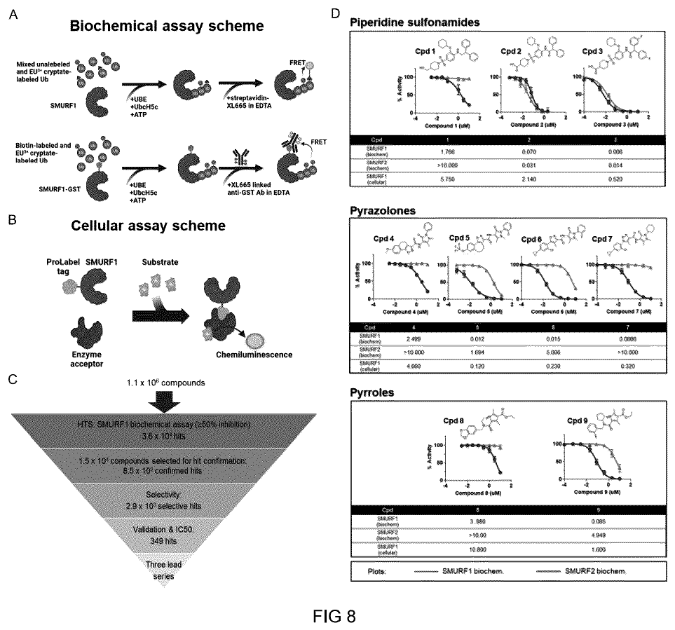

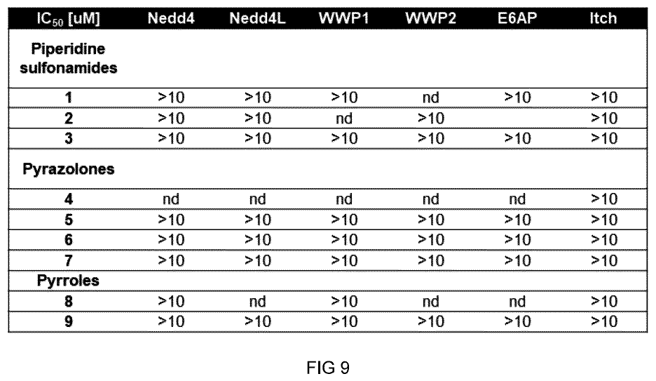

To overcome the lack of a known active site pocket, the disclosers developed a time-resolved fluorescence resonance energy transfer (TR-FRET)-based assay reporting SMURF1 self-ubiquitylation (FIG. 8A). Using this system, the disclosers undertook a large, unbiased high-throughput screen (HTS) of 1.1 million compounds. Primary hits were further screened using biochemical selectivity and cell-based assays designed to prioritize and optimize molecules for specific SMURF1 inhibition (FIGS. 8B and 8C). This process revealed three chemical series with favorable drug-like properties: piperidine sulfonamides, pyrazolones, and pyrroles (FIG. 8D and FIG. 9). Although SMURF1 and SMURF2 share a high degree of protein sequence homology (86% identity), the disclosers identified potent inhibitors that were selective for SMURF1 over SMURF2 (FIG. 8D and FIG. 9), which were structurally distinct from those reported in prior studies.

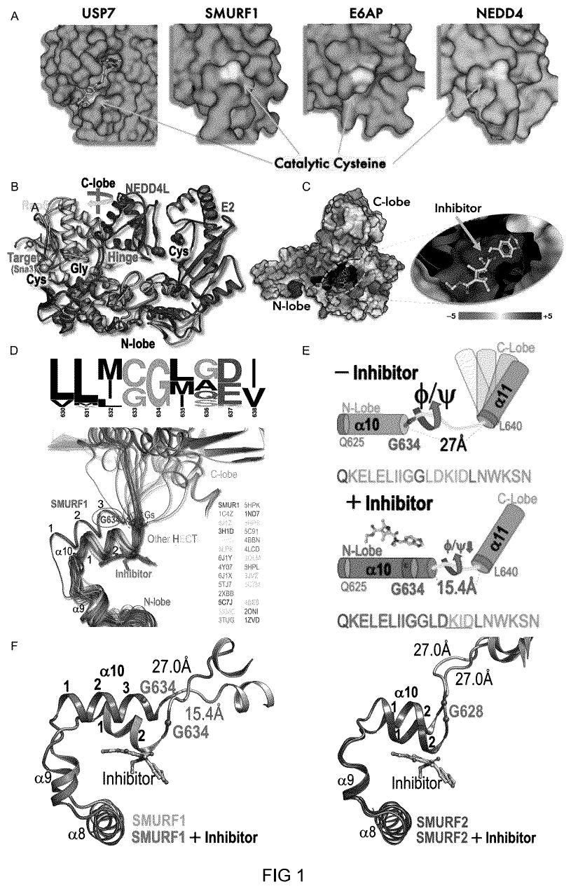

To gain structural insights into the mechanism of selective inhibition of SMURF1, the SMURF1 and SMURF2 HECT domains were crystalized without and with a representative SMURF1 inhibitor compound #8 (Cpd. 8) and the structures were determined at 2.05-2.75 Å resolution (FIGS. 1B-D). These studies presented the first structure of the SMURF1 HECT domain. In the absence of an inhibitor, SMURF1 and SMURF2 HECT domains adopt a highly similar architecture, comprising N- and C-lobes connected by a flexible glycine-containing hinge (residues G634-D639, FIGS. 1B-D). The superimposed HECT domain structures of all family members suggest that the motion between the N- and C-lobes around this hinge is important for ubiquitin transfer from E2 to the target protein via the HECT C-lobe. The range of this motion is defined by the conformation adopted at two extremes: the ubiquitin˜E2˜HECT complex and the ubiquitin˜HECT˜target complex. Function of the hinge is facilitated by its length and the flexibility provided by amino acid residues with a tolerance to a wide range of ϕ/ψ dihedral angles (FIGS. 1D-1E).

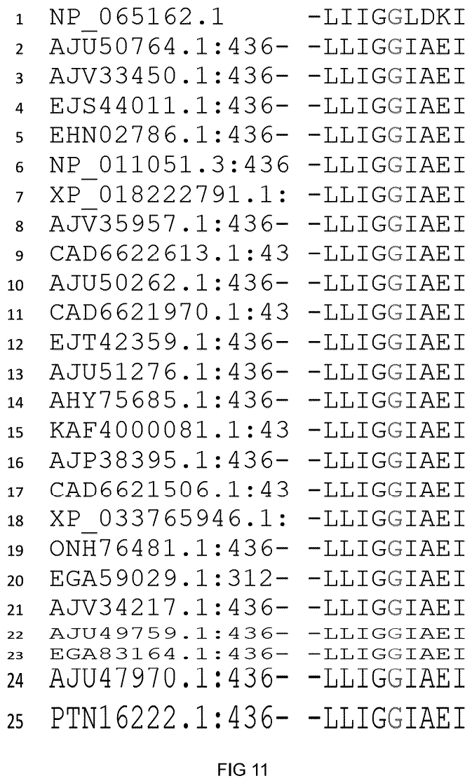

Highly conserved amino acid sequences outside of active sites pockets and conserved domains are often of critical importance to the structure and function of a protein. To determine amino acid sequences critical for regulating SMURF1 activity, a Fast Fourier transformation based multiple alignment was employed. These analyses demonstrate that the G634 residue, which stems the SMURF1 hinge, is invariant in all HECT sequences in the NCBI protein bank across animal, plant and fungal kingdoms and all 28 HECT domain structures (FIG. 1D and FIG. 11). The functional importance of this invariant glycine is reinforced by the UBE3A/E6AP G738E153 mutation which causes Angelman syndrome.

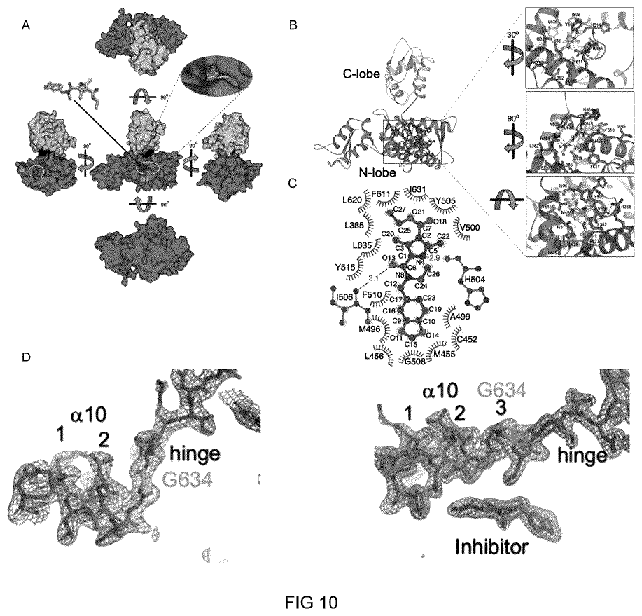

Across all 28 HECT domain structures, the a-helix #10 (aH10) comprises two and a half turns and ends with the conserved glycine (FIG. 1D). In complex with SMURF1, the inhibitor is buried in an allosteric binding site in the N-lobe, with only 2% of its surface exposed to solvent (FIGS. 10A-C). Compared to unbound SMURF1, αH10 of the SMURF1-inhibitor complex is elongated by one and a half turns over the conserved glycine (G634). This shortens the hinge (from 27.0 Å to 15.4 Å) and replaces G634 at the stem of the flexible hinge with lysine (K637), an amino acid with a lower tolerance of the ϕ/ψ dihedral angles (FIG. 1D-E). These crystallographic data were confirmed by unbiased refinement using electron density with Sigma-A mFo-DFc simulated-annealing omit-maps (FIG. 1F, and FIG. 10).

The structure of SMURF2 in complex with Cpd-8 demonstrated binding of the same allosteric binding site as the SMURF1-inhibitor complex. However, in contrast to SMURF1, inhibitor binding did not elongate the SMURF2 αH10, and the invariant glycine (G628) stems the hinge in both the inhibitor-bound and -unbound forms (FIG. 1F). Consistent with the absence of allosteric structural changes, SMURF2 activity was not reduced by inhibitor binding (FIG. 8).

These data indicate that binding of the allosteric SMURF1 inhibitor reduces flexibility and length of the hinge thereby preventing the required motion between the N- and C-lobes to transfer ubiquitin from the E2 to the target suggesting that this allosteric mechanism is responsible for the allosteric activity of the inhibitor and enzyme inhibition.

### SMURF1 Escape Mutants Resist Allosteric Inhibition

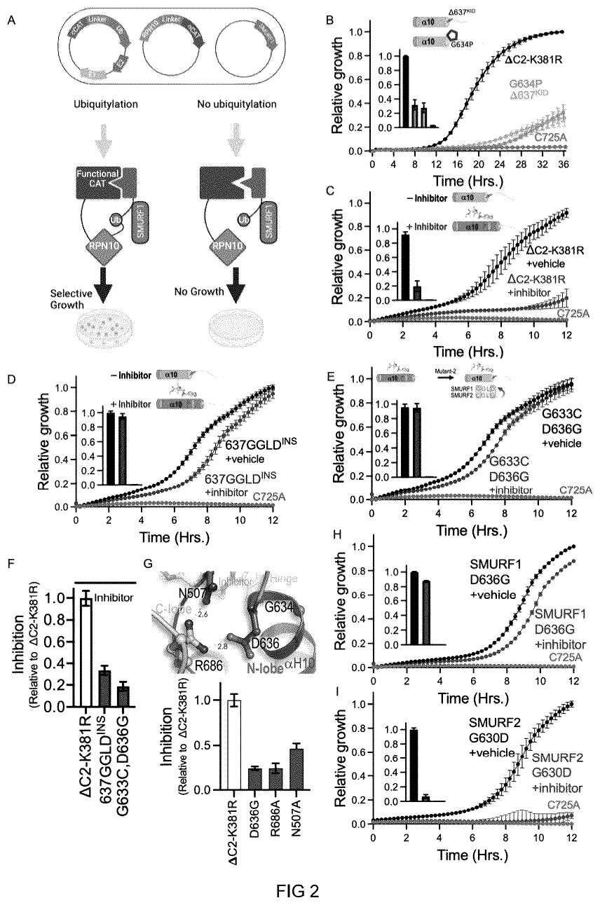

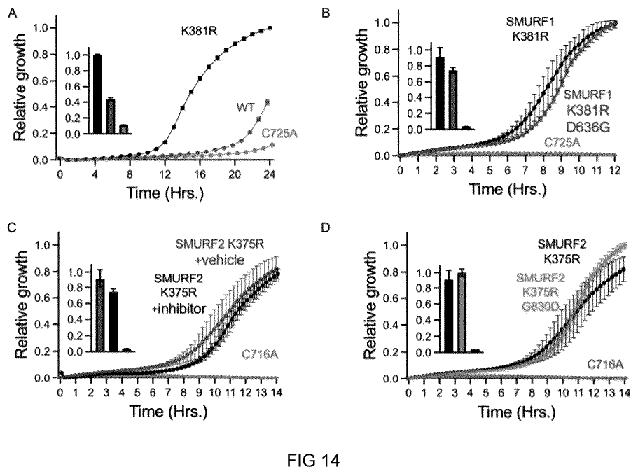

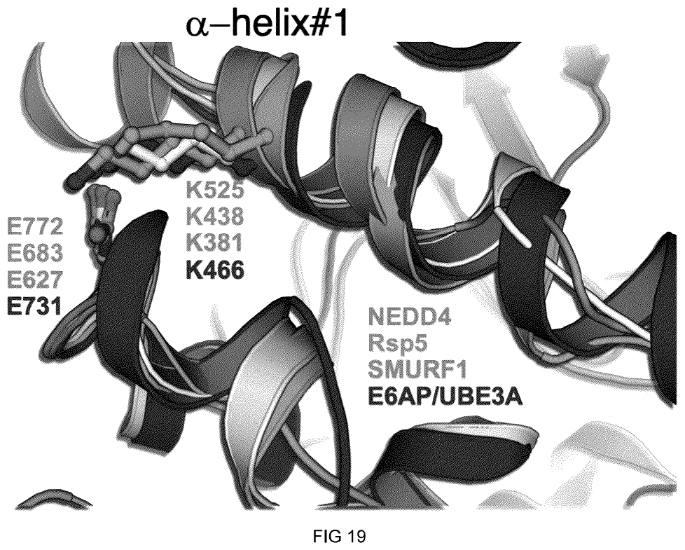

To biochemically test the structure-based hypothesis of the SMURF1 glycine hinge function, the disclosers constructed mutants that either restrict the ϕ/ψ dihedral angles or shorten the hinge length. The disclosers employed an E. coli split chloramphenicol acetyltransferase (split-CAT) system as a reporter to measure SMURF1 activity using Rpn10, a protein that is efficiently ubiquitylated by all E3-ligases in vitro, as a target (FIGS. 2A and 14). In this assay, ubiquitylation results in functional assembly of the split-CAT, allowing for selective bacterial growth, which serves as a quantitative indicator of ubiquitylation efficiency (FIG. 2A). To maximize the assay amplitude, the disclosers constructed a hyperactive ΔC2-K381R SMURF1 mutant that lacks the self-ubiquitylation-dependent regulation reported for NEDD4/Rsp5 (FIG. 19). When expressed in the split-CAT-based E. coli reporter system, the hyperactive ΔC2-K381R SMURF1 mutant increased Rpn10 ubiquitylation compared to both the wild-type SMURF1 and the catalytically inactive C725A SMURF1 (FIG. 14, ΔC2-K381R SMURF1 and ΔC2-K375R SMURF2 are used as the basis for all further mutation and inhibition assays).

To investigate the importance of hinge flexibility and length for SMURF1 function, two classes of mutants were constructed: (1) a mutant with reduced hinge flexibility, created by substituting the conserved glycine with proline (G634P), which reduces ϕ/ψ angles due to a covalently linked side chain; (2) a mutant with reduced hinge length, created by deleting three amino acids. Both mutants reduced ubiquitylation activity, confirming the critical role of glycine hinge flexibility and length (FIG. 2B).

Further tests showed that Cpd-8 significantly attenuated the activity of SMURF1 (FIG. 2C). The disclosers therefore engineered two SMURF1 mutants designed to resist the effect of the allosteric inhibitor: (1) a mutant that restores the flexibility and length of the hinge (637GGLDINS), constructed by inserting a GGLD sequence immediately downstream of the elongated αH10 induced by inhibitor binding (FIG. 2D); (2) a mutant that maintains inhibitor binding but resists allosteric αH10 elongation, constructed by replacing two critical residues at the C-terminal end of αH10 with those present in SMURF2 (G633C, D636G, FIG. 2E). Neither 637GGLDINS nor the G633C, D636G double mutant significantly altered SMURF1 activity in the absence of the inhibitor. Notably, both mutants retained more than 75% activity in the presence of Cpd-8, indicating that preservation of the glycine hinge flexibility and length provides effective escape from the inhibitor (FIGS. 2D-2F).

### A Lock Mechanism Stabilizing Elongated αH10

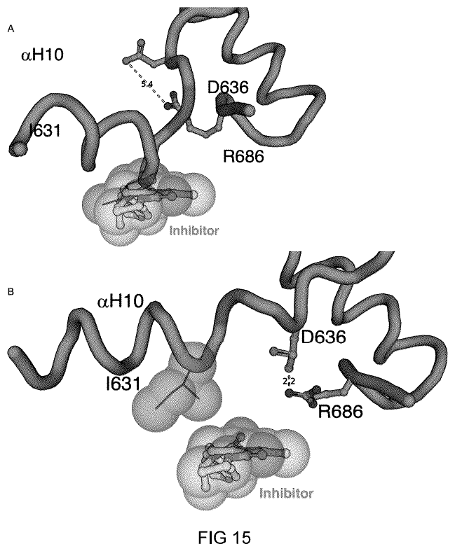

Further structural analyses suggested that a network of non-covalent bonds between D636, R686, and N507 stabilizes the allosterically elongated αH10 of SMURF1, induced by compound binding (FIG. 2G). Based on this observation, the disclosers modeled the transition of the αH10 and glycine hinge from the unbound to the compound-bound state (FIG. 15). The analysis showed that in unbound SMURF1, I631 of the αH10, along with other nearby residues, occupies the allosteric binding site, preventing small molecule inhibitor binding. In the presence of Cpd-8, the αH10 is displaced, inducing allosteric elongation. In this altered conformation, an electrostatic bond is formed between the αH10 D636 and C-lobe R686. R686 is further stabilized by N507, which is allosterically shifted toward R686.

To evaluate the importance of this network of non-covalent bonds, SMURF1 mutants were generated, and enzymatic function was assessed against a phospho-mimetic SMAD1 peptide target in the bacterial system. The independent mutations D636G, R686A, and N507A did not alter enzymatic activity but conferred resistance to inhibition by Cpd-8, thereby demonstrating the importance of αH10 stabilization of the inhibited form of SMURF1 (FIGS. 2G and 2H and 14B).

SMURF1 R686 is conserved in SMURF2 as R680, but SMURF1 D636 is replaced with G630 in SMURF2, meaning that the network of non-covalent bonds stabilizing the elongated αH10 in SMURF1 cannot be formed in SMURF2. Consistent with this, SMURF2 was not inhibited by Cpd-8 (FIG. 14C). To examine whether introducing the residues required for formation of the stabilizing bond network would render SMURF2 sensitive to inhibition, the disclosers engineered SMURF2 with a G630D substitution. Activity of the G630D SMURF2 against the phospho-mimetic SMAD1 peptide target was not significantly altered (FIG. 14D). However, the G630D SMURF2 was susceptible to inhibition by Cpd-8 (FIG. 2I).

Together, these data confirm that binding of the inhibitor to SMURF1 induces elongation of the αH10 over a conserved glycine hinge, stabilized by a network of non-covalent bonds, which reduces flexibility and length of the glycine hinge that is essential for catalytic action. The resistance of SMURF2 to inhibition is likely conferred by G630, which, in the presence of the compound, does not form the bond network required to lock the elongated αH10.

### A Mechanism Applicable to Pyrazolone and Pyrrole Compounds

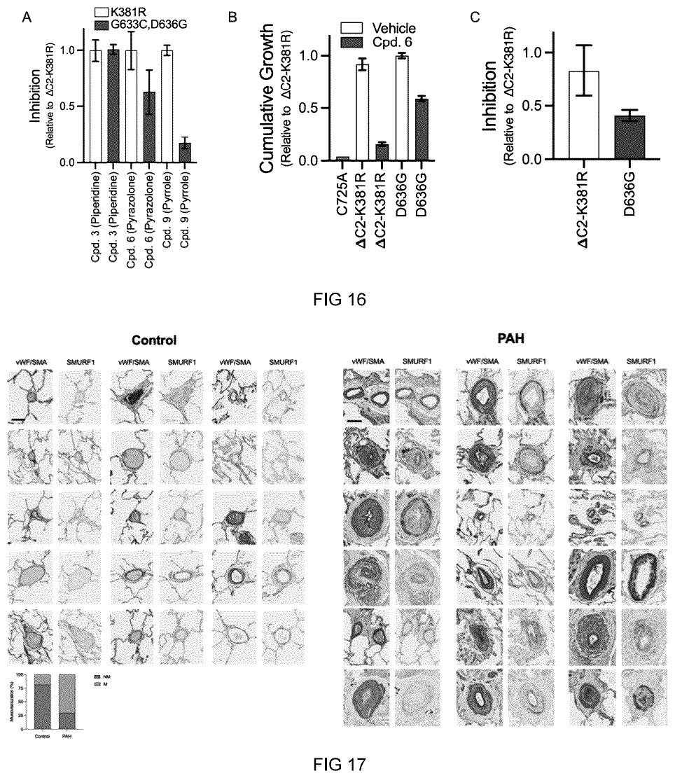

The allosteric mechanism described was proposed based on the crystal structures of SMURF1 and SMURF2 with and without Cpd-8, and validated in the split-CAT system with the same compound. The disclosers next sought to investigate whether compounds from each of the three chemical series identified in the unbiased screen function through the same allosteric mechanism. Using the split-CAT system, the disclosers found that Cpd-3 (piperidine), Cpd-6 (pyrazolone) and Cpd-8 (pyrrole) all inhibited ubiquitylation corroborating the findings from the TR-FRET assay. However, in SMURF1 mutants engineered to increase the flexibility and length of the glycine hinge, only Cpd-6 and Cpd-8 showed reduced inhibition. This confirmed that Cpd-6 and Cpd-8 function through the identified allosteric mechanism, while Cpd-3 functions via an alternate mechanism (FIG. 16A). This finding is consistent with the specificity of the compounds in that Cpd-3 inhibits both SMURF1 and SMURF2 whereas Cpd-6 and Cpd-8 inhibit only SMURF1 (FIG. 8D). To gain structural insights into the mechanism of non-selective inhibition, the disclosers crystalized the HECT domain of SMURF2 without and with Cpd-2, and determined structures at 2.05-2.75 Å resolution. Cpd-2 bound the same cryptic pocket between the C- and N-lobe of SMURF2 as Cpd-8. However, in contrast to the Cpd-8, Cpd-2 extended from the pocket to physically interact with the C-lobe of SMURF2 preventing the required motion of the glycine hinge without allosteric elongation αH10. Due to its drug-like properties, the disclosers further evaluated the mechanism of inhibition of Cpd-6 using SMAD1, a native SMURF1 target. By demonstrating that escape mutants resisted inhibition, the disclosers confirmed that Cpd-6 functions through the same allosteric mechanism as Cpd-8 (FIGS. 16B and 16C).

### SMURF1 is Overexpressed in PAH

Human genetic studies implicate reduced BMP signaling as an initiating factor in the onset of PAH. Ligand binding of the BMP receptor complex releases TRIB3, which negatively regulates SMURF1 (FIG. 3A) thereby reducing degradation of key mediators of the BMP pathway, including SMAD1. As such, BMP signaling increases, and reduced BMP signaling reduces, availability of downstream signaling mediators through SMURF1. SMURF1 expression is also increased by hypoxia or TGFβ signaling, both of which are central drivers of vascular remodeling (FIG. 13), further implicating SMURF1 as a key mediator of reduced BMP signaling and the subsequent vascular remodeling observed in PAH. To understand the effect of disease-associated mutations on SMURF1 regulation, the disclosers engineered HEK293 cells to stably express GFP-tagged SMURF1. Consistent with prior reports, BMP4 stimulation reduced the GFP-tagged SMURF1 levels (FIG. 3B). However, following siRNA-mediated reduction of ACVRL1, BMPR2 and ENG, the level of GFP-tagged SMURF1 increased in response to BMP4 stimulation (FIG. 3B). PAH is characterized by remodeling of endothelial and smooth muscle cells of the small pulmonary arteries, in which increased SMURF1 expression has been described. To investigate the penetrance of this finding, the disclosers examined SMURF1 expression in small pulmonary arteries in 33 PAH patients. The disclosers observed significantly increased SMURF1 expression in endothelial and smooth muscle cells in all samples from PAH patients compared to 19 patients without PAH. This is the first report of SMURF1 expression in PAH-affected lungs at the cellular level, using the largest sample size to date. Thus, this disclosure represents the most comprehensive assessment of SMURF1 expression in the lungs of PAH patients (FIGS. 3C and 3D, and FIG. 17).

### SMURF1 Directly Ubiquitylates SMAD1 and BMPR2

BMPR2 and SMAD1 are key mediators of BMP signaling, both of which are reduced in patients (FIG. 3A). SMURF1 has been reported to directly ubiquitylate SMAD1, but studies suggest that BMPR2 ubiquitylation is mediated through SMAD6 or SMAD7. SMAD1 and the intracellular domain of BMPR2 were expressed in the split-CAT-based E. coli selection system to assess if each were direct targets of SMURF1 (FIG. 4A). Compared to wild-type SMURF1, the hyperactive SMURF1 increased ubiquitylation of both targets, while the C725A catalytic inactive mutant reduced ubiquitylation (FIGS. 4B and 4C). These results are corroborated by the AlphaFold model of the SMURF1:BMPR2 interaction, suggesting that two WW domains, but not the C2 domain of SMURF1, interact with BMPR2. As E. coli lacks the accessory proteins that bridge SMURF1 to BMPR2, these data demonstrated that BMPR2 is a direct target of SMURF1. To evaluate the relevance of this finding to mammalian cells, SMAD1 and BMPR2 were overexpressed in HEK-293 cells with key components of the ubiquitylation cascade. Inhibition of SMURF1 increased levels of both SMAD1 and BMPR2 (FIGS. 4D and 4E). Consistent with the proposed allosteric mechanism of inhibition of SMURF1, mutations designed to reduce the length and flexibility of glycine hinge (G634P and Δ637KID) increased SMAD1 levels. Mutations designed to preserve hinge length and flexibility (G633C, D636G double mutant and 637GGLDINS) enabled escape from inhibition (FIG. 4D). Together, these data demonstrate allosteric inhibition of SMURF1 in mammalian cells and, through its direct targets BMPR2 and SMAD1, modulation of two critical points within the BMPR2 pathway.

### SMURF1 Inhibition Augments BMP Signaling

To investigate the role of allosteric SMURF1 inhibition on BMP signaling in disease-relevant cells, pulmonary artery smooth muscle cells (PASMCs) from patients with idiopathic and heritable PAH were treated with BMP4 and cultured in the presence of a SMURF1 inhibitor or vehicle. Compared to vehicle control, allosteric inhibition of SMURF1 led to increased levels of BMPR2, unphosphorylated SMAD1/5/8, phosphorylated SMAD1/5/8, and ID1 and increased activation of the BMP response element (FIG. 4F and FIG. 12A). These findings demonstrate that SMURF1 inhibition augments BMP signaling in pulmonary vascular cells.

### SMURF1 Inhibition Re-Establishes Pulmonary Vascular Homeostasis

Modulation of BMP signaling, either through genetic or pharmacological manipulation, is known to alter key cellular phenotypes implicated in the pathology of PAH. BMP9 is the predominant BMP ligand that regulates pulmonary vascular endothelial homeostasis and acts protectively against vascular remodeling. Consistent with the identified increase in BMP signaling in human primary pulmonary artery endothelial cells (PAEC), the disclosers found that SMURF1 inhibition effectively reduced apoptosis and proliferation of these cells in the presence of a low BMP9 concentration (FIGS. 4G-4I). Similarly, in the presence of BMP4, the predominant ligand that regulates pulmonary vascular smooth muscle homeostasis, SMURF1 inhibition reduced proliferation and migration of primary pulmonary artery smooth muscle cells (PASMC) from patients with PAH (FIGS. 4J-4M). This effect was observed in the presence and absence of BMPR2 mutation. To determine the on-target effects of allosteric SMURF1 inhibitors on the global proteome in a relevant disease-specific cellular context, PASMCs from patients with PAH were pre-treated with BMP4 under hypoxic conditions and exposed to the SMURF1 inhibitor or a vehicle (FIGS. 12B and 12C). Effective inhibition was demonstrated by increased protein levels of the established SMURF1 targets RhoA and TGFBR1 and key components of the BMP signaling pathway SMURF1, BMPR2, SMAD1, pSMAD1, and ID1 (FIG. 12D). Gene set enrichment analysis showed that SMURF1 inhibition modulated multiple processes implicated in disease, including TGFβ superfamily (including BMP signaling) and IL-1β signaling, extracellular matrix (ECM) remodeling, and fibrosis (FIG. 12E).

### SMURF1 Inhibition Reverses Established, Experimental PAH Pathology

Based on molecular and cellular data, the disclosers hypothesized that administering an allosteric SMURF1 inhibitor would restore BMP signaling in animal PAH models with reduced BMPR2 expression. Pulmonary hypertension was induced in Wistar Kyoto rats through treatment with monocrotaline (MCT) or SU5416 under hypoxic conditions (FIGS. 5A and 5F). Twenty-one days after MCT administration, and twenty-eight days after the administration of SU5416 under hypoxic conditions (10% O2), right ventricular systolic pressure and pulmonary artery remodeling were increased (FIGS. 5B, 5C, 5G, and 5H). Consistent with the demonstrated augmentation of BMP signaling, allosteric inhibition of SMURF1 improved right ventricular systolic pressure (FIGS. 5B and 5G) and pulmonary artery remodeling (FIGS. 5D, 5E, 5I, and 5J) in a dose-dependent manner. To assess the potential toxicity of allosteric SMURF1 inhibitors, a detailed biochemical and histopathology assessment was performed on blood and organs from the MCT study (FIGS. 5A-5E). No toxicologically relevant findings were observed. Taken together, these data demonstrate that allosteric SMURF1 inhibitors effectively treated experimental PAH and exhibit minimal toxicity.

### Leveraging SMURF1 Allosteric Mechanism to Inhibit E6AP

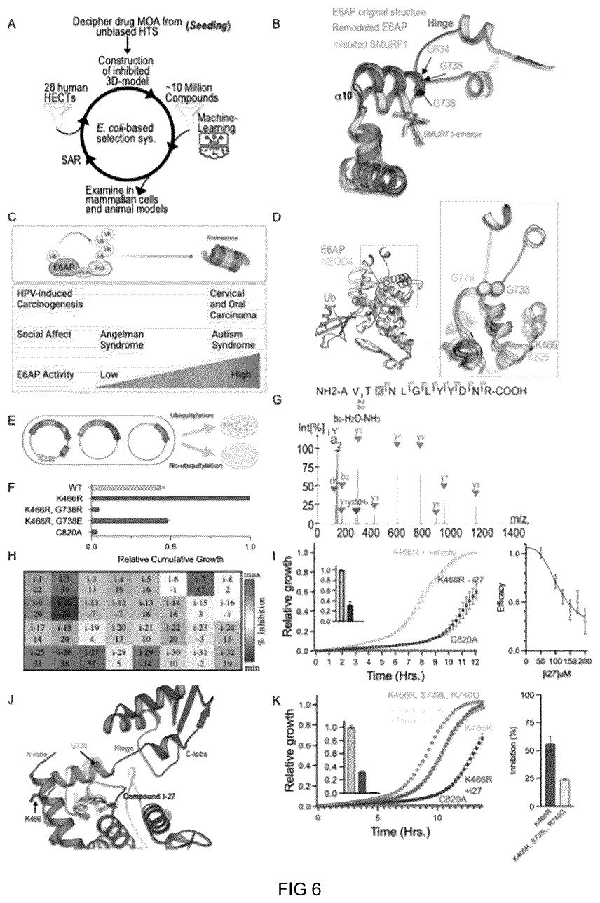

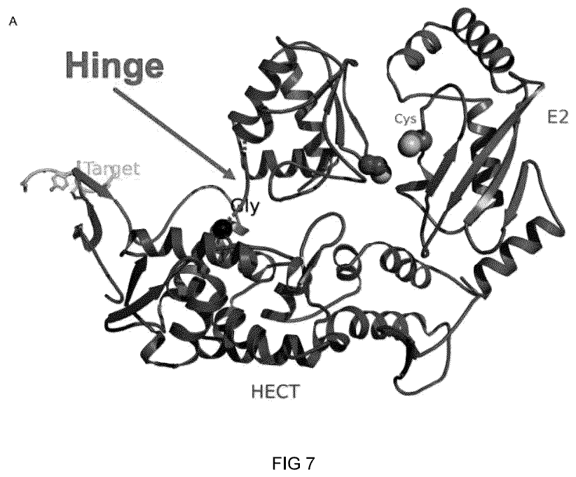

As the SMURF1 inhibitor specifically restrains a conserved, essential motion between the N- and C-lobes of the HECT domain, the disclosers hypothesized that similar allosteric inhibition could be used as the basis of an in-silico method for identifying potential inhibitors of other HECT E3-ligases (FIG. 6A). To model a potential binding cavity, the disclosers threaded the amino acid sequence of HECT family members onto the structure of SMURF1 in its inhibited state (FIG. 6B). The model was characterized by an elongated αH10 that traverses the conserved glycine residue, a shortened hinge between the N- and C-lobes, and an open cavity in the N-lobe. The disclosers then employed a machine learning-based screen to filter ˜8 million molecules, selecting candidates with the highest scores for downstream assessment.

To assess this approach and demonstrate its broad applicability, the disclosers investigated the prototypic HECT E3 ligase E6AP, which is implicated in diseases including human papillomavirus (HPV)-associated cervical and oropharyngeal cancers, sleep disorders, Angelman syndrome, and autism. Mutation of the conserved αH10 glycine in E6AP (G738E) reduces activity and causes Angelman syndrome. E6AP is expressed from the maternally imprinted UBE3A gene. Increased E6AP levels and/or through aberrant paternal expression, duplication, triplication, or decreased degradation all lead to autism (FIG. 6C).

The AlphaFold model of full-length E6AP indicated that K466 spatially corresponds to K525 in human NEDD4, K438 in yeast Rsp5, and K381 in SMURF1. This lysine residue is critical to self-ubiquitylation-dependent oligomerization and subsequent inactivation (FIGS. 6D and 19). Mutation of K466 is linked to autism and oropharyngeal cancer and is predicted to constitutively hyperactivate the enzyme. To confirm the structural importance and demonstrate the capacity of the split-CAT reporter assay to detect alteration in E6AP-mediated ubiquitylation, the disclosers introduced a K466R mutation, which significantly increased ligase activity (FIGS. 6E and 6F). Further substantiating this finding, mass spectrometry analysis, undertaken using a semi-tryptic peptide search approach to identify anticipated K-GG-modified peptides, revealed self-ubiquitylation at K466 of protein purified from E. coli expressing the full ubiquitylation apparatus of E6AP. This finding is consistent with re-analysis of datasets containing diGly peptides from human cell lines (HEK293 and U2OS) which demonstrated K466 ubiquitylation in E6AP. Together, these data support the concept of oligomerization-dependent inactivation of E6AP and provide a critical link to human disease (FIG. 6G).

The mutations G738E/R associated with Angelman syndrome (corresponding to SMURF1 G634) significantly decreased E6AP activity in the split-CAT system (FIG. 6F). These two mutants provide, for the first time, structural mechanistic explanations for the E6AP mutations associated with cancer, autism, and Angelman syndrome.

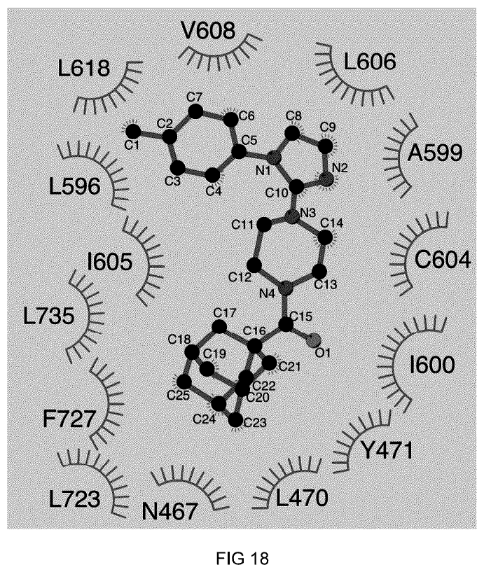

The disclosers employed the split-CAT E6AP reporter system to select potential inhibitors from the in-silico screening hits. Of 32 examined compounds, three demonstrated reduced E6AP activity by ≥39% (FIG. 6H) without alteration of CAT activity or E. coli toxicity. Compound i-27 reduced E6AP activity in a dose-dependent manner (FIGS. 6I and 6J, and 18), without affecting E1, E2, or CAT activities, suggesting selective inhibition. To determine if compound i-27 functions through the proposed allosteric mechanism, the Ser-Arg residues located immediately downstream of E6AP-G738 in the glycine hinge were substituted with the corresponding residues of SMURF2 (S739L, R740G). The disclosers found that the S739L, R740G double mutant did not reduce E6AP activity. In the presence of compound i-27, inhibition was reduced twofold, demonstrating escape from inhibition (FIG. 6K). Together, these results confirm the disclosers hypothesis that computational screening for inhibitors that stabilize a remodeled, inhibited structure can identify compounds functioning through the same allosteric mechanism. Moreover, the described process of remodeling an inhibited enzyme having an allosteric binding site may provide a valuable strategy for discovering other allosteric inhibitors, including HECT ligases (FIG. 6A).

### Discussion

To date, about 3,000 distinct drugs have been approved for clinical use. Moreover, it is estimated that collectively, they target just 3% (˜650 proteins) of the ˜20,000 human gene products. Protein kinases, phosphodiesterases, ion channels, G-protein coupled receptors and nuclear hormone receptors represent the majority of druggable protein classes owing to presence of specific druggable pockets. However, many other proteins remain undrugged. With 22% of the proteome estimated to be disease modifying, there remains a clear opportunity to expand the druggable space and identify new treatments for human diseases. Importantly, the 28 human HECTs are attractive therapeutic targets without a confined druggable active-site pocket.

The present disclosure provides an innovative approach that mitigates a critical challenge in the development of inhibitors to protein targets without an apparent active-site pocket. A traditional unbiased screening flow-chart using an advanced combination of biochemical, biophysical and cellular assays combined with automation to enable the discovery of the described allosteric inhibitors. While in silico screens based on available structures provide a significant advance to drug discovery programs, the present disclosure underscores the importance of traditional, unbiased approaches in facilitating discovery for enzymes without traditional or obvious binding pockets and the synergistic nature of the two strategies.

### The Flexible (Glycine) Hinge: a Critical, Druggable Protein Structure?

Around 30% of human proteins contain a hinge enabling motion between domains critical for function. Hinge flexibility requires amino acid residues with a large tolerance of F and Y dihedral angles, typically glycine and serine, and relies on weak interactions of the mobile domain with other domains within the same protein or residing protein complex. HECT inhibition, by limitation of the essential motion between the N- and C-lobes, is a natural biological phenomenon which regulates enzymatic activity. Restriction of motion may be mediated in several ways including: by the C2 domain of several Nedd4 members (an effect that is itself reversed by phosphorylation), by regions around the WW domains, or an α-helix that is inserted between the HECT's lobes; or through oligomerization-dependent inactivation. The identification HECT inhibitors that leverage a natural allosteric mechanism to limits hinge flexibility provides a framework for the design of small molecules that modify the function of other proteins that require the flexibility provided by this structure such as ion channels, immunoglobulins, and connexins.

### Therapeutic HECT E3 Inhibition for the Treatment of Human Disease

A single ubiquitin ligase typically modulates the levels and functions of multiple related protein targets through proteolytic and nonproteolytic mechanisms. These post-transcriptional modifications act in concert to exert coordinated effects on cellular signaling and cell function. E6AP, the prototypical HECT E3 ligase, is central to the pathology of HPV-related cancer, and Angelman and neurodevelopmental syndromes. Mutations of E6AP G738 cause Angelman syndrome and, as demonstrated, lead to reduced activity through restriction of the glycine hinge. The present biochemical disclosure also demonstrates the importance of E6AP K466 self-ubiquitylation mutation which is associated with abnormalities of neurodevelopmental syndromes. For the first time, a structural and mechanistic explanation for this phenomenon is provided, which aligns with the observed decrease in ligase activity in human disease.

The Mendelian association of mutations within the BMP/TGFb superfamily and PAH provides a strong rational for the modulation of related signaling pathways to treat disease. Augmentation and/or modulation of the BMP pathway has been shown to provide benefits in experimental models of PAH. Here, it is demonstrated that SMURF1 is increased in the pulmonary vasculature of patients with PAH and that direct SMURF1-mediated ubiquitination of BMPR2 and SMAD1/5/8 leads to target degradation, thereby further implicating SMURF1 in the pathology of disease. Supporting the translational potential of this finding, it is shown that SMURF1 inhibition restores levels of BMPR2 and SMAD1/5/8 and augments pathway signaling to restore pulmonary vascular function and treat experimental PAH. This finding provides deeper understanding of disease pathology and potential therapeutics for this field. A number of therapies focused on rebalancing BMP/TGFβ pathways in patients with PAH are in clinical development, the most advanced of which, an activin receptor IIa ligand trap, has been shown to provide clinical benefit. A key differentiating feature of SMURF1 inhibition as a therapeutic strategy is the direct augmentation of the pathway receptor and downstream signaling mediators which directly addresses the receptor deficiency present in human disease. The development and availability of multiple therapeutic tools that modulate these pathways offers the opportunity to dissect the pathology of disease and mechanisms of therapeutic response facilitating approaches to personalized therapy. Together, the present disclosure demonstrates that augmentation of BMP signaling may offer a transformative, novel approach for the treatment of PAH.

In summary, important implications of the reported findings include (1) the identification of a novel mechanism for specific HECT E3 ligase inhibition and other proteins with a glycine hinge domain (2) identification of a novel inhibitor binding site through an unbiased biochemical screen of 1.1 million compounds, which in silico screens targeted to a putative candidate binding pocket would not have yielded (3) the facilitated investigation of BMP pathway augmentation as a novel strategy for the treatment of patients with PAH through the specific inhibition of SMURF1 (4) a strategy for identification and optimization of selective E3 ligase inhibitors with suitable drug-like properties offering clinical potential (5) novel insight into the importance of the E6AP glycine hinge domain in HPV-related cancer and Angelman's and autism syndrome and (6) a targeted approach to inhibit HECT E3 ligases enabling faster identification of hits for lead optimization. This is the first selective SMURF1 HECT domain small molecular weight inhibitor with demonstrated activity in disease animal models and suitable drug-like properties offering an urgently needed new therapy for PAH patients and potentially multiple other diseases where SMURF1 is implicated. The deciphered mechanism of action of the inhibitor offers a new class of drugs for HECT E3-ligases and other glycine-hinge containing proteins, opening a new druggable space with potential to address diseases urgently in need of transformative new therapies.

According to certain embodiments, the HECT E3-ligase inhibitor can include piperidine sulfonamides which are a class of compounds characterized by the incorporation of a piperidine ring bonded to a sulfonamide group; pyrazolones which are a class of compounds featuring a pyrazolone ring structure; or pyrroles, characterized by a five-membered aromatic ring containing one nitrogen atom.

### Methods

### Biochemical SMURF1 High Throughput Biochemical Assay and E3 Ligase Selectivity Panel

The SMURF1 HTS was conducted as a FRET assay in which the donor lanthanide fluorophore is europium, encapsulated by cryptate for stability, and the acceptor fluorophore is a cross-linked allophycocyanin (XL665). When both fluorophores are in proximity through the ubiquitylation reaction, the excited europium cryptate emits light at 590 nm, the energy is transferred to XL665, which releases a fluorescence signal at 665 nm. In the primary screen, the polyubiquitylation of SMURF1 was detected with a mixture of wild type europium-labelled ubiquitin and biotinylated-ubiquitin, which elicited a signal upon the addition of streptavidin labelled XL665 (FIG. 8). For validation screening, the interaction between the GST-tagged SMURF1 and the mixture of wild type ubiquitin and europylated-ubiquitin elicited a signal that was proportional to the level of polyubiquitylation upon addition of anti-GST XL665 (FIG. 8). A ratiometric readout of 665 nm/590 nm was taken to reduce some of the assay variation. The experimental conditions of the HTS correspond to the SMURF1 assay described in detail for the selectivity panel.

### E3 Ligase Selectivity Assay Panel

E3 ligase specificity assay panel employed the same TR-FRET principle. Compounds were tested for HECT E3 ligase selectivity in an assay panel consisting of the following ubiquitin E3 Ligases: SMURF1 (aa 119-757), SMURF2 (aa 251-748), WWP1 (aa 546-922), WWP2 (aa 494-870), Nedd4 (aa 619-1000), Nedd4L (aa 693-1074), Itch (aa 433-903) and E6AP (aa 2-852). All E3 ligases were expressed as N-terminal HA-tagged fusion proteins in E. coli. (except for full length E6AP which was expressed in baculo virus infected insect cells as a N-terminal GST-fusion protein).

For compound testing, serial dilutions were prepared in DMSO and 50 nl transferred to the assay plates (384-Well-Plate “SMALL VOLUME”, 30 ul-Well). To these assay plates 4.5 μl E3 ligase solution was added per well followed by 4.5 μl of the pre-incubated mix containing E1a, E2 and biotinylated ubiquitin mix or the pre-diluted ubiquitin (control) in 50 mM HEPES, pH 7.5, 50 mM NaCl, 10 mM MgCl2, 20 μM ATP, 0.1 mM DTT, 0.002% Triton X-100. The final assay concentrations of the components were 3-30 nM E3 ligase, 20 nM E1a, 50 nM E2 (UbCH5b for SMURF1/2, UbCH7 for Itch, E6AP, UbCH5c for WWP1/2 and Nedd4/4L) and 20 nM biotinylated Ub or the unmodified ubiquitin (control).

After 45 min of incubation at RT the ubiquitylation reactions were stopped by adding 4.5 μl STOP solution (2 mM NEM, 50 mM HEPES, pH 7.5, 150 mM NaCl, 0.01% Triton™ X-100) immediately followed by 4.5 μl of detection solution including the XL665-labeled antibody and the streptavidin-coupled europium (20 nM Streptavidin Europium, 40 nM XL665-labeled anti-HA or anti-GST antibody, 50 mM HEPES, pH 7.5 150 mM NaCl, 0.01% Triton X-100) to give a total volume of 18.0 μl. After a further incubation of 45 min at RT in the dark, the plates were transferred into a fluorescence reader to measure the TR-FRET signal. A ratiometric readout of 665 nm/590 nm was taken to reduce some of the assay variation.

### Cellular Compound Screening Assay to Assess SMURF1 Inhibition

The DiscoverX PathHunter™ technology was used to determine Prolabel-tagged SMURF1 protein levels in an enzyme complementation assay of the cell lysate. HEK293 cells were stably transfected with SMURF1 N-terminally fused to the Prolabel tag (a fragment of beta-galactosidase) which complements the exogenously added enzyme fragment to form functional beta-galactosidase. Upon incubation with small molecules, a chemiluminescent signal is generated by addition of a substrate to the cell lysate. The signal is proportional to SMURF1 protein levels (FIG. 8).

To assess SMURF1 inhibition and subsequent stabilization a stable HEK293 cell pool expressing a Prolabel-SMURF1 fusion protein was generate via lentiviral transduction. The Prolabel tagged SMURF1 construct was inserted into the p40 lentiviral vector (confers puromycin resistance) and virus was prepared. HEK293 cells were seeded into a 6-well plate at 2×105 cells per well the day before infection. The next day the medium was exchanged with fresh DMEM high glucose, 10% FBS, PenStrep with 10 μg/ml Polybrene. 100 μl of the ProLabel-SMURF1 lenti virus preparation were added to the medium. Cells were incubated overnight, the medium was replaced by fresh polybrene-free medium for 24 Hrs before the medium was replaced with fresh medium containing puromycin (2 μg/ml final concentration) to start the selection of clones with integrated SMURF1. The cells were incubated in selection medium until all cells in the un-transfected control plate had died. The puromycin-resistant HEK293_Prolabel hSMURF1 cells were expanded in puromycin-containing selection medium and frozen down in aliquots for further use.

For compound testing, serial dilutions were prepared in DMSO and 250 nl transferred to the assay plates (384 well plates). Cells were detached from the flask after a short incubation with trypsin-EDTA, counted and diluted to a concentration of 0.75×106 cells/ml in culture medium without puromycin. The expression of SMURF1 is induced by adding doxycycline to a final concentration of 0.2 μg/ml. 20 μl of the cell suspension are seeded into the compound plates by using the MULTIDROP™ 384. The plates are incubated over night at 37° C., 5% CO². After overnight incubation with the compounds, the levels of SMURF1 are determined using the PathHunter Prolabel detection kit (Cat #: 93-0180L, DiscoverX). First 10 μl of a lysis/CL detection working solution are added, followed by the addition of 5 μl enzyme acceptor EA. The plates are mixed on a plate shaker and incubated for 2-3 Hrs at RT before measuring the chemiluminescent signal in the PheraStar plate reader for 1 sec/well.

### BMP Reporter Gene Assay (RGA)

To measure BMP signaling, a BMP reporter assay was established using a stable cell line containing a reporter gene construct encoding luciferase (luc) proximal to the BMP Response Element (BRE). Cells expressing the BMP reporter were co-incubated with SMURF1 inhibitor and 10 ng/ml of BMP4 for 12 Hrs. BRE activity was determined by Bright-Glo™ (E1500, Promega™) as per manufacturer's instructions.

### Cellular Assay to Determine SMURF1 Stability Using SMURF1-GFP

To measure SMURF1 protein abundance, a stable HEK293 cell line was generated using a vector encoding SMURF1 protein with C-terminal GFP (RG222902). HEK293 cells were transformed as per standard protocols briefly, after plasmid preparation and Maxiprep, cells were transfected with 2.5 μg of plasmid DNA using 1, 1.5, 2 μL of Lipofectamine LTX as per manufacturer's instructions and GFP fluorescence measured via Incucyte S4. Optimal transfection conditions were determined by maximal GFP fluorescence. After sorting, cells were seeded and cultured for 4-6 wks with appropriate antibiotic selection. GFP fluorescence was measured against time on the Incucyte S4 and signal after 12 Hrs of compound incubation quantified as experimental endpoint. For RNA knock-down experiments, siRNA was delivered using Lipofectamine 3000 and the following probes administered (all purchased from ThermoFisher™, USA): ACVRL1 (Assay ID: VHS41062, Cat #1299001), BMPR2 (Assay ID: S2044, Cat #4390824), ENG (Assay ID: S4677, Cat #4392420), SMAD9 (Assay ID: S8415, Cat #4392420) as per the manufacturer's instructions. SMURF1 protein level was determined by GFP florescence read via Incucyte S4.

### Crystallography

SMURF1 was crystallized by the hanging drop vapor diffusion method. 1 μL SMURF1 at a concentration of 15.1 mg/ml in 50 mM Tris pH 8.0, 200 mM NaCl, 2 mM TCEP and 10% glycerol were mixed with 1 μL reservoir solution (25% PEG 3350, 0.1 M Bis Tris pH 5.9, 0.2 M MgCl2, 0.02 M NH4OAc) and equilibrated against 600 μL reservoir solution.

SMURF2251-748 was crystallized by the hanging drop vapor diffusion method. 2 μL SMURF2 at a concentration of 10.7 mg/ml in 50 mM Tris pH 8.0, 20 mM NaCl and 2 mM TCEP were mixed with 1 μL reservoir solution (1.5 M NaH2PO4/K2HPO4 pH 6.8, 100 mM NaOAc) and equilibrated against 1 mL reservoir solution. Crystals were soaked by the addition of 0.5 μL inhibitor stock solution (100 mM inhibitor in 90% DMSO) to the crystal containing drop for 30 mins.

Co-crystals of SMURF1 in complex with inhibitor 8 were grown by the sitting drop vapor diffusion method. 0.2 μL SMURF1 at a concentration of 15.1 mg/mL SMURF1 in 50 mM Tris pH 8.0, 20 mM NaCl, 2 mM TCEP, 2 mM inhibitor 8 and 1.8% DMSO was mixed with 0.3 μL reservoir solution (25% PEG1500, 100 mM SPG pH 9) and equilibrated against 80 μL reservoir solution.

For data collection, crystals were flash cooled in liquid nitrogen. X-ray diffraction data were collected from single crystals at the Swiss Light Source, beamline X06DA equipped with a MAR225CCD detector for SMURF1 and beamline X10SA equipped with a Pilatus Pixel detector for crystals of SMURF1 and SMURF2 in complex with inhibitor 8. The diffraction data were processed and scaled with the autoPROC toolbox. The structures were solved by molecular replacement using the coordinates of PDB code 1zvd as search model and the program MOLREP82. For structure solution of SMURF1 the search model was split in the C- and N-lobe and the hinge residues and the helix N-terminal to the hinge were omitted from the search model. The software programs COOT83 and BUSTER™ (Global Phasing™ Ltd. Cambridge, UK) were used for iterative rounds of model building and structure refinement. Images were generated using the program PyMOL (DeLano Scientific™ LLC, San Carlos, CA, USA). PDB codes: SMURF1 apo structure: 8C52, SMURF1—compound 8 complex: 8C55 SMURF2—compound 8 complex: 8C5A.

### Bacterial E3 Ligase Assay (Split-Chloramphenicol Acetyl Transferase Assay for Ubiquitin Ligase Activity)

Assays were performed as previously described.84,85 The selection plasmids were co-transformed into Mach1™-T1R E. coli cells. All growth assays were performed in LB agar or liquid at 37° C. Optimization of chloramphenicol (CAM) concentrations were performed according to the largest growth shift between the hyperactive mutant to the catalytic dead mutant in SMURF1 (ΔC2-K381R; ΔC2-C725A) or in E6AP (HECTd-K466R; HECTd-C820A) at each assay as previously described. The disclosers constructed a split-CAT based E3-dependent Rpn10 full length ubiquitylation cascade to monitor the effect of inhibitor and point mutations in ubiquitin E3 ligases. Specifically, yeast Rpn10 was tethered to N-CAT as a general ubiquitylation target and co-expressed with C-CAT-Ub, E1, E2 and human SMURF1235-757: wild-type DC2, K381R or catalytic dead C725A. Similarly, E6AP453-852: wild-type HECTd, K466R or catalytic dead HECTd-C820A.

To inspect ubiquitylation of BMPR2, the disclosures substituted Rpn10 with BMPR2174-558 cytosolic portion fused to the N-CAT. To characterize the mechanism of action of the SMURF1 inhibitor, the disclosers used the hyperactive mutant of SMURF1, DC2-K381R as a platform to other mutations in the ligase (G634E/P, 633GGLD>CGLG and insertion of GGLD downstream to D636) and test their activity where Rpn10 served as a general ubiquitylation target. Accordingly, the disclosers used the hyperactive mutant E6AP HECTd-K466R for other mutations in the ligase (G738E/R, 737CGSR>CGLG). Inhibition assays were performed in liquid LB using 96 well-plate with Tecan Sunrise reader high intensity shaking for 9.5 mins rest for 10, 5 mins shake and read at 595 nm. Growth was monitored between 12-36 Hrs as indicated.

### PASMC Assays

Primary human pulmonary arterial smooth muscle cells from patients with PAH underdoing transplant were obtained from approved tissue banks in Royal Papworth Hospital Research Tissue Bank, UK (Research ethics: 08/H0304/56+5).

Proliferation: 17 Cells were seeded at 5,000 per well in 96 well plates (3595, Corning, USA) in full growth media (DMEM with 10% low Ig FBS and 5 ml of pen/strep, Gibco, USA) and allowed to adhere then swapped into starvation media (DMEM with 0.2% low Ig FBS) for 24 Hrs to synchronize the cells in a non-proliferative state. Cells were cultured in the indicated concentration of growth media and stimulated with BMP4 (314-BP-010, R&D, USA) with increasing concentrations of SMURF1 inhibitor to determine effect on cellular proliferation using phase microscopy to determine cell confluence on the Incucyte S4 live cell imager with packaged analysis software. Confluence was determined along the entire time course by expressing total cell numbers per well to maximum cell number obtained by culturing cell in full growth media.

Migration: cells were seeded at 3,000 per well in 96 well plates in full growth media (DMEM with 10% low Ig FBS and 5 ml of pen/strep, Gibco) and allowed to adhere. Migration, in the presence of indicated growth media, stimulant (BMP4, 10 ng/ml final, 314-BP-010, R&D) and compound was assessed by disc closure as per manufacturer's instructions (CBA-126, Cell Biolabs™) using phase microscopy to determine migration on the Incucyte™ S4 live cell imager with packaged analysis software. Disc size was determined at 4, 8 and 12 Hrs following removal of the biocompatible hydrogel.

Cell signaling studies: cells were seeded at equal densities, allowed to adhere, and then incubated in starvation media for 24 Hrs to synchronize cells in a non-proliferative state in the presence of DMSO or 5 μM SMURF1 inhibitor. Cells were stimulated with a concentration of BMP4 (10 ng/ml) found to elicit a sub-maximal response in absence of SMURF1 inhibitor to initiate signaling. 2 Hrs after addition of BMP4, cells were harvested and lysed. Specific proteins were quantified using the Protein Simple Western Blot System (Wes) (ProteinSimple™, CA, USA). The following primary antibodies were used and paired with appropriate secondary antibodies for detection: SMURF1 (H00057154-M01, Abnova™, USA), BMPR2 (orb69398, Biorbyt, UK), SMAD1/5/9 (ab66737, Abcam™, USA), phospho-SMAD1/5 (9516S, Cell Signaling Technology, USA), ID1 (M085, CalBioreagents™, USA), GAPDH (2118L, Cell signaling Technology™, USA) or beta actin (4967, Cell Signaling Technology, USA).

### PAEC Assays

Primary human pulmonary arterial endothelial cells (PAECs) (CC-2530, Lonza, USA) were seeded in 96 well plates (3570, Corning™, USA) at 5,000 per well in full growth media (CC-3162, Lonza™, USA) and allowed to adhere.

Apoptosis: 30 16 Hrs before the addition of an apoptotic stimulus, PAEC were transferred into EBM-2 basal media (Lonza, USA) with 2% FBS, 100 U/ml penicillin, 100 mg/ml streptomycin and 0.25 mg/ml amphotericin B in the presence of indicated growth media, stimulant (BMP9, 10 ng/ml final, 3209-BP-010, R&D, USA) and compound/DMSO. Following incubation, cells were either left unstimulated or treated with 10 ng/ml TNFα and 20 μg/ml cycloheximide to induce apoptosis which was assessed by presence of Annexin V as per manufacturer's instructions (JA1000, Promega™, USA).

Proliferation: 30 Cells were seeded at 5,000 per well in 96 well plates in full growth media and allowed to adhere. Cells were cultured in the indicated concentration of growth media and stimulated with (BMP9, 3209-BP-010, R&D, USA) with increasing concentrations of SMURF1 inhibitor to determine effect on cellular proliferation using phase microscopy to determine cell confluence on the Incucyte S4 live cell imager with packaged analysis software. Confluence was determined along the entire time course by expressing total cell numbers per well to maximum cell number obtained by culturing cell in full growth media.

### Proteomic Analysis

PASMCs from PAH patients carrying either mutant BMPR2 (C347Y) or WT allele were cultured to 80-90% confluence then swapped into starvation media for 24 Hrs to synchronize cells in a non-proliferative state in the presence of DMSO or SMURF1 inhibitor compound (Cpd 6, 5 μM) in normoxic or hypoxic conditions. Hypoxic cells were cultured in 1% O2 at 37° C. for 5 days prior to cell harvest. All media changes were performed with media that had been incubated in hypoxic conditions for 24 Hrs to ensure no re-oxygenation. Cells were stimulated with BMP4 (10 ng/ml) to initiate signaling and prior to cell harvest for protein isolation and quality control by quantification via Protein Simple Western Blot System.

Treated PASMCs were collected, washed three times with ice-cold PBS (Cat #: 20012-027, Thermo Fisher, USA) and transferred as frozen cell pellets to perform tandem mass tag (TMT)-based expression proteomics analysis as previously described.87 Cells were lysed with 500 μl of lysis buffer (8 M urea, 1% SDS and 50 mM Tris, pH 8.5, with protease and phosphatase inhibitors added) and sonicated to shear DNA aggregates. Once centrifugation was completed, the protein concentrations were measured by following a Micro BCA Protein Assay kit (Cat #: 23235, Thermo Fisher™). 300 μg of protein was aliquoted from each sample and reduced with 5 mM DTT for 1 h at room temperature, alkylated with 15 mM iodoacetamide for 1 Hr at room temperature in the dark and then quenched with 10 mM DTT for 15 min at room temperature. Alkylated proteins were purified via chloroform-methanol precipitation,88 resuspended in denaturing buffer (8 M urea and 50 mM Tris, pH 8.5) and diluted with seven volumes of 50 mM Tris, pH 8.5. Protein was digested using Trypsin-Lys-C mix (Cat #: A40009, Thermo Fisher, USA) in an enzyme: protein ratio of 1:25 and incubated overnight at 37° C. A second digestion was performed with additional Trypsin-Lys-C mix (enzyme:protein ratio of 1:50) for 5 Hrs. Peptides from each sample were then desalted using a Water's tC18 SepPak plate (Cat #: 186002321, Waters, USA), dried down and resuspended in 100 μl of 0.1 M TEAB buffer, pH 8.5.

For each sample, 200 μg of peptides were labeled via TMT11plex Isobaric Label Reagent kit (Cat #: A34808, Thermo Fisher, USA) at the ratio of four units of TMT reagent to one unit of peptide. Once the TMT labeling efficiency was confirmed by MS analysis to be greater than 99%, the reaction was quenched with 0.5% with hydroxylamine for 15 min at room temperature. Equal amounts of each TMT-labeled sample were combined, desalted using Water's tC18 SepPak plate (Cat #: 186002321, Waters, USA) and fractionated by HPLC using a Waters XBridge C18 (3.5 μm, 300×4.6 mm) column with gradient of 10-40% mobile phase B (90% acetonitrile with 5 mM ammonium formate, pH 10) in mobile phase A (5 mM ammonium formate with 2% acetonitrile). Final fractionated peptide material was pooled into 24 fractions (˜1-2 μg of peptides per fraction). Each fraction was analyzed using an Orbitrap Fusion Lumos mass spectrometer (Thermo Fisher, USA) equipped with a Reprosil-PUR column (1.9-μm beads, 75-μm ID×15-μm tip×20 cm, 120 Å). Samples were run using gradients of 6-28% mobile phase B (80% acetonitrile with 0.1% formic acid) in mobile phase A (0.1% formic acid) using the SPS MS3 mode.

Raw mass spec data files were processed by ProteomeDiscoverer™ (PD) ver. 2.4 (Thermo Fisher, USA). Searches were conducted using the PD Sequest node against Uniprot human canonical database (downloaded in July 2019 containing 21,482 sequences including common mass spec contaminants) with the following parameters: 10 ppm precursor tolerance, MS2 fragment ion tolerance of 0.6 Da, static modifications of TMT(+229.163 Da) on lysine and peptide N-terminal, and carbamidomethylation of cysteine residues(+57.021 Da), while oxidation of methionine residues(+15.995 Da) was set as a variable modification, and three missed cleavages were allowed. Peptide spectrum matches were filtered using SPSmatch>60, precursor interference<50, average TMT S/N>10, and FDR determined by PD Percolator node<1%. Shared peptides were used to assemble peptides to proteins and protein groups but were excluded from quantitative analysis. TMT intensities were corrected for isotopic impurities based Thermo Fisher provided QC sheet specific to TMT batch used in the analysis. Resulting table with TMT intensities were exported and processed with internally developed R script that filtered to protein FDR of 1% for each TMT plex, and normalized TMT intensities across four TMT11 plexes using CONSTANd procedure.

Given high donor to donor biological variability of cell lines, instead of using mean values of protein abundances across treatment groups (n=2), protein changes were visualized for individual donors where cells derived from the same donor were utilized as treatment and controls, FIG. 11. Only proteins quantitated in both donors were used for analysis, 7,632 of total 8,666 protein quantitated in either donor. To evaluate protein dysregulation, R package clusterProfiler90 as used to carry out enrichment analysis with MetabaseMap™ (GeneGO™, Clarivate™, Philadelphia, PA) and MSigDB (Broad Institute™, Cambridge, MA) geneset collections. Uniprot primary accessions were converted to ENTREZ ID using the org.Hs.eg.db R package. Both up and down regulated proteins in either donor were determined by selecting proteins outside of confidence area (ellipse) at 0.9999 level as calculated by ggplot package(˜400 proteins). A list of all identified proteins was used as “universe of possible proteins” to estimate significance of enriched terms, and default cut-offs (pval<0.01 and qval<0.2) were applied to select significantly enriched gene sets.

The raw mass spectrometry proteomics data is available in ProteomeXchange Consortium via the PRIDE91 partner repository with the dataset identifier (To be included upon publication).

### In Vivo Models

All studies described in this report were performed according to UK Animals (scientific procedures) Act 1986. Monocrotaline model: 17 Animals (male Wistar Kyoto, Harlan, UK) were injected with a sub-cutaneous dose of monocrotaline (Crotaline™, Sigma™, Poole, UK) 60 mg/kg (0.5 ml/kg). On day 14 animals were dosed with 0.5 ml test agent or vehicle orally (p.o.) for 7 days either once or twice per day as indicated.

Sugen/hypoxia model: 92 animals (male Wistar Kyoto™, Harlan, UK) were injected with a sub-cutaneous dose of 20 mg/kg (1 ml/kg) Sugen (SU5416, Sigma™, Poole, UK) before being caged in hypoxic conditions (10% O2, normobaric with constant humidity and CO2 levels, 24 Hrs/day) for 14 days. Animals were then returned to normoxia (21% O2) and test agents administered via oral gavage (0.5 ml), q.d. for a further 14 days. All SMURF1 inhibitors were prepared in 0.5% methyl cellulose: 0.5% Tween 80 and Imatinib prepared in sterile water. At study end, animals were anaesthetized using ketamine/medetomidine, the jugular vein was surgically exposed and blood flow isolated with a distal ligature. A small hole was made in the vessel and a 2F Millar pressure/volume catheter introduced and progressed into the right ventricle (RV), where an average RV pressure was measured during systole (RVSP). The lungs were excised from the rats and inflated with 10% neutral-buffered formalin then immersed in 10% neutral-buffered formalin to complete fixation for 24-48 Hrs.

### Histology for Human Specimens

Formalin-fixed, paraffin-embedded lung sections from 32 patients with PAH and 19 non-PAH patients undergoing transplant were obtained from approved tissue banks in Royal Papworth Hospital Research Tissue Bank, UK (Research ethics: 08/H0304/56+5) and Paris, France (CPP EST-III n° 18.06.06, Le Kremlin-Bicêtre, France). All patients gave informed consent.

### Dual Immunohistochemistry on Human Specimens

A dual immunohistochemical assay was applied using the Ventana Discovery Ultra platform (Roche Diagnostics™, USA). Sequential incubation and detection of primary antibodies were applied after heat induced epitope retrieval at pH 8.5. vWF (1:500 dilution, Cat #A0082, Dako) followed by Omni Map anti-rabbit HRP (Cat #460-4311, Roche Diagnostics, USA) and visualized by addition of DAB. Next anti-α-SMA Ab (1:16,000 dilution, Cat #A2547, Sigma) was added to the same slide and detected by addition of anti-mouse NP, anti-NP AP conjugate and visualized by administration of Discovery Red chromogen (all reagents: Roche Diagnostics, USA). Slides were scanned using an Aperio Scanscope AT (Leica Biosystems, IL, USA).

### Co-Localization Immunohistochemistry on Human Specimens

A bright field dual marker co-localization method was established to determine localization of SMURF1 relative to α-SMA and separately on serial slides from the same tissue blocks, SMURF1 and vWF and performed on human histology specimens using the Ventana Discovery Ultra platform (Roche Diagnostics, USA). Firstly, serial slides were stained for each of the individual markers. Sequential incubation and detection of primary antibodies were applied after heat induced epitope retrieval at pH 8.5. SMURF1 detection was performed by administration of anti-SMURF1 antibody (1:800 dilution, Cat #AB57573, Abcam, USA) followed by incubation with anti-mouse HRP conjugated secondary (Omni Map anti-mouse HRP Cat #760-150, Roche Diagnostics, USA) and visualized with Purple Chromogen (Cat #760-229, Roche Diagnostics, USA). For vWF detection, anti-vWF antibody was applied (1:500 dilution, Cat #A0082, Agilent-Dako, USA) followed by anti-rabbit conjugated to NP (Cat #760-4817, Roche Diagnostics, USA) and anti-NP antibody (Cat #760-4827, Roche Diagnostics, USA) and visualized by addition of Yellow Chromogen (Cat #760-239, Roche Diagnostics, USA). For α-SMA detection, anti-α-SMA Ab (1:16,000 dilution, Cat #A2547, Sigma-Aldrich, USA) was added to the same slide and detected by addition of anti-mouse NP conjugated secondary Cat #760-4816, Roche Diagnostics, USA) followed by addition of anti-NP antibody (Cat #760-4827, Roche Diagnostics™, USA) and visualized by administration of Yellow Chromogen (Cat #760-239, Roche Diagnostics, USA). Next, serial sections were co-stained for SMURF1+vWF and separate serial slides co-stained for SMURF1+α-SMA using the same antibody and protocols described for the single stains. Specificity of staining was determined by use of isotype controls applied to serial sections from selected blocks: for SMURF1 (1:800 dilution, isotype mouse IgG2a Cat #70-4724, Tonbo Biosciences™, USA), α-SMA (1:1430 dilution, isotype mouse IgG2a Cat #70-4724, Tonbo Biosciences, USA) and vWF (1:1760 dilution, isotype rabbit IgG Cat #I-1000, Vector Labs™, USA). Co-localization assays required denaturation before the incubation of the second primary antibody to prevent non-specific cross reactivity. Slides were scanned using an Aperio™ Scanscope AT. Where two antibodies co-localize, a color shift to red is evident.

### Immunohistochemistry on Rodent Specimens

Monocrotaline model: FFPE slides were dual immuno-stained using the Ventana Discovery Ultra platform (Roche Diagnostics, USA). Sections were dewaxed, and antigens were retrieved using CC1 (pH 8.5) and incubated for 16 mins. Dual immuno-staining was performed by sequential incubation of the same slide with first anti-vWF antibody (1:500 dilution, Cat #A0082, Agilent-Dako, USA) followed by detection with anti-rabbit-HRP conjugated secondary (Rabbit Omni Map HRP Cat #460-431, Roche Diagnostics, USA) and visualized via ChromoMap DAB (Cat #760-159 Roche Diagnostics, USA). Secondly, anti-α-SMA antibody was applied to the same slide (1:16,000 dilution, Cat #A5228, Sigma-Aldrich™, USA) and detected with anti-mouse NP conjugated secondary (Cat #760-4816, Roche Diagnostics, USA) and visualized via Discovery Red (Cat #760-228, Roche Diagnostics, USA). Specificity of staining was confirmed by absence of staining of slides from representative blocks with the same isotype controls at the same dilutions as used for human sections described above. Slides were scanned using an Aperio XT slide scanner. Arteriole remodeling following monocrotaline exposure was measured by counting 100 small vessels(<100 um diameter) and assigning each vessel as either non-muscularized (no α-SMA staining), partially muscularized, or fully muscularized (thick unbroken wall of smooth muscle), and then the percentage distribution of each was calculated per group. The histological analyst was blind to the experimental conditions.

Sugen/hypoxia model: Lungs were processed and dual α-SMA/vWF immunohistochemistry performed as described for the monocrotaline model. Arteriole remodeling was measured by image analysis software (ImagePro, Media Cybernetics, UK), which quantified the α-SMA positive pixels as a measure of percent muscularization of selected vessels<100 μm in diameter.

Phosphorylated (p)SMAD1/5/8: Staining was performed using Ventana as described above. pSMAD1/5/8 was detected by incubation with anti-pSMAD1/5/8 antibody (1:50 dilution, Cat #9511, Cell Signaling Technologies, USA) followed by incubation with HRP-conjugated polyclonal swine anti-rabbit secondary (1:200, Cat #E03553, Dako-Agilent, USA).

### In Silico Screening for E6AP Allosteric Inhibitors

Remodeling allosteric inhibited E6AP. HHblits was used to find up to 500 homolog proteins for the HECT domain of E6AP and SMURF1 in uniref30 database. Redundant sequences with identity higher than 90% were removed using mmseq2 and the obtained profiles were aligned with MAFFT. The protein alignment was manually inspected and adjusted to ensure that conserved residues were aligned correctly. This alignment was used to instruct modeler to build a structural model of E6AP based on the inhibited conformation of SMURF1. To remove any steric clashes from the model, Prime energy minimization was used with the OPLS-4 force field and VGSB solvation model. Minimization was performed in iteration, each with 65 steps, or until converging to a 0.01 Kcal/mol/Å gradient.

### Ligand Preparation and Virtual Screening

6.4 million commercially available, drug-like molecules were downloaded from the ZINC database. The ligands were prepared using Ligprep (Schrödinger LLC, NY). Stable protonation and tautomerization states at 7.0±2 pH were generated for each molecule by Epik. Chirality was maintained as it were in ZINC. Virtual screening was performed in two phases using Glide molecular docking. First, the entire compound library, including all generated isoforms, was docked using the low accuracy, HTVS docking mode. Second, the top-ranking 10th percentile, were redocked using the more rigorous SP docking mode. The top ranking thousand ligands from the second phase of the docking were visually inspected and, based on their binding pose, 32 compounds were selected, purchased and used for experimental verification in the E6 AP Split-CAT E. coli system.

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

### Definitions

In silico method: A computational method or approach performed using comparative data and computers to model, analyze, or predict biological structures and processes, such as enzyme structure or protein-ligand interactions, based on comparisons with other genetic or amino acid sequences, biophysical structures, amino acid bonding, allosteric changes and other physical, chemical, or biological conditions, states, or changes that can be modeled on a computer. The in silico methods can include those known in the art and those identified herein. In some cases, in silico method can include implementation of AI and ML.

Small molecule allosteric inhibitor: A low molecular weight compound that binds to an allosteric site of a protein, distinct from the catalytic site, causing a structural or conformational change in the protein that reduces or inhibits its catalytic activity. In some cases, the allosteric site or cavity is a cryptic cavity.

Target protein family: A group of evolutionarily related proteins that share conserved structural or functional features, including similar domains or active sites, and are typically involved in analogous biological processes. In some non-limiting embodiments, the target protein family can include the family of HECT E3-ligases or members thereof.

Template protein member: A representative protein from the target protein family that serves as a structural and functional reference for modeling, due to its known genetic and amino acid sequence, hinge domain, allosteric binding site, catalytic site and activity. The template protein member serves a model protein for in silico modeling to screen for enzymes that have similar allosteric inhibition from similar small molecule allosteric inhibitors due to similar features. In some non-limiting embodiments, the template protein member can be a template HECT E3-ligase such as SMURF1 or selected from a closed group of HECT E3-ligase enzymes.

Hinge domain: A flexible structural region of a protein that facilitates structural or conformational changes necessary for its function, often located between its N-terminal and C-terminal domains. According to some embodiments, the hinge domain comprises a glycine-hinge.

Allosteric binding site: A site on an enzyme, spatially distinct from the catalytic site, where small molecule allosteric inhibitors can bind to induce a conformational or structural change that alters the enzymes activity, either enhancing, inhibiting, upregulating, or downregulating its function.

Catalytic site: The active region of an enzyme responsible for facilitating biochemical reactions by binding substrates and converting them into products.

Allosteric change: A conformational or structural alteration in a protein induced by the binding of a molecule to an allosteric site, resulting in modulation of the protein's function or activity.

Inhibited structure: A structural state of a protein in which its catalytic activity is reduced or inhibited, such as a result of binding by an allosteric inhibitor.

Threading corresponding amino acid sequences: An in silico process of aligning and mapping amino acid sequences and structures from different proteins onto a known structural framework, such as that of a template protein, to generate a model that reflects conserved features and structures.

Model target protein: A computationally generated representation of a target protein, incorporating structural and sequence features derived from a template protein and related family members. The model target protein can be selected and/or designed to have specific conserved amino acid residues, protein structures, allosteric binding sites and activity, enzymatic activity, catalytic site, and mechanism of inhibition.

Small molecule allosteric inhibitor candidates: A set of compounds identified through computational or experimental screening that are predicted to bind to the allosteric binding site of a target protein and potentially act as allosteric inhibitors based on molecular composition, structure, charge, size, or other physical or chemical features similar to known allosteric inhibitors of the template protein member.

Non-covalent bonds: Non-covalent interactions are critical in maintaining the three-dimensional structure of large molecules, such as proteins and nucleic acids. The term encompasses electrostatic bonds, pi bonds, van der Waals forces, and hydrophobic effects.

### Sequence Listings

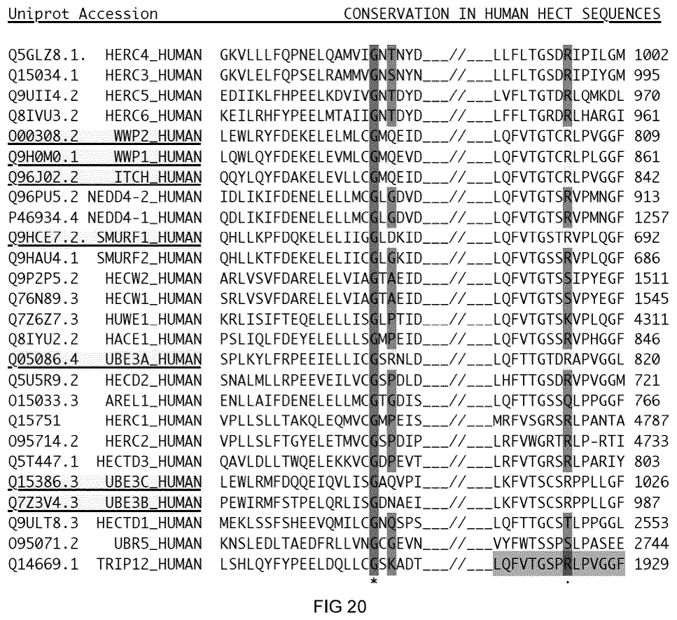

TABLE 1
Sequence Listing. Conserved amino acids in
the respective sequences are shown in FIG. 20.

| Seq. | | |
|---|---|---|
| ID | Enzyme/Uniprot | |
| No. | Accession | Amino Acid Sequence |
| 1 | Q5GLZ8.1. HERC4_HUMAN | GKVLLLFQPNELQAMVIGNTNYD |
| 2 | Q5GLZ8.1. HERC4_HUMAN | LLFLTGSDRIPILGM |
| 3 | Q15034.1 HERC3_HUMAN | GKVLELFQPSELRAMMVGNSNYN |
| 4 | Q15034.1 HERC3_HUMAN | LLFLTGSDRIPIYGM |
| 5 | Q9UII4.2 HERC5_HUMAN | EDIIKLFHPEELKDVIVGNTDYD |
| 6 | Q9UII4.2 HERC5_HUMAN | LVFLTGTDRLQMKDL |
| 7 | Q8IVU3.2 HERC6_HUMAN | KEILRHFYPEELMTAIIGNTDYD |
| 8 | Q8IVU3.2 HERC6_HUMAN | LFFLTGRDRLHARGI |
| 9 | O00308.2 WWP2_HUMAN | LEWLRYFDEKELELMLCGMQEID |
| 10 | O00308.2 WWP2_HUMAN | LQFVTGTCRLPVGGF |
| 11 | Q9H0M0.1 WWP1_HUMAN | LQWLQYFDEKELEVMLCGMQEVD |
| 12 | Q9H0M0.1 WWP1_HUMAN | LQFVTGTCRLPLGGF |
| 13 | Q96J02.2 ITCH_HUMAN | QQYLQYFDAKELEVLLCGMQEID |
| 14 | Q96J02.2 ITCH_HUMAN | LQFVTGTCRLPVGGF |
| 15 | Q96PU5.2 NEDD4-2_HUMAN | IDLIKIFDENELELLMCGLGDVD |
| 16 | Q96PU5.2 NEDD4-2_HUMAN | LQFVTGTSRVPMNGF |
| 17 | P46934.4 NEDD4-1_HUMAN | QDLIKIFDENELELLMCGLGDVD |
| 18 | P46934.4 NEDD4-1_HUMAN | LQFVTGTSRVPMNGF |
| 19 | Q9HCE7.2. SMURF1_HUMAN | QHLLKPFDQKELELIIGGLDKID |
| 20 | Q9HCE7.2. SMURF1_HUMAN | LQFVTGSTRVPLQGF |
| 21 | Q9HAU4.1 SMURF2_HUMAN | QHLLKTFDEKELELIICGLGKID |
| 22 | Q9HAU4.1 SMURF2_HUMAN | LQFVTGSSRVPLQGF |
| 23 | Q9P2P5.2 HECW2_HUMAN | ARLVSVFDARELELVIAGTAEID |
| 24 | Q9P2P5.2 HECW2_HUMAN | LQFVTGTSSIPYEGF |
| 25 | Q76N89.3 HECW1_HUMAN | SRLVSVFDARELELVIAGTAEID |
| 26 | Q76N89.3 HECW1_HUMAN | LQFVTGTSSVPYEGF |
| 27 | Q7Z6Z7.3 HUWE1_HUMAN | KRLISIFTEQELELLISGLPTID |
| 28 | Q7Z6Z7.3 HUWE1_HUMAN | LQFVTGTSKVPLQGF |
| 29 | Q8IYU2.2 HACE1_HUMAN | PSLIQLFDEYELELLLSGMPEID |
| 30 | Q8IYU2.2 HACE1_HUMAN | LQFVTGSSRVPHGGF |
| 31 | Q05086.4 UBE3A_HUMAN | SPLKYLFRPEEIELLICGSRNLD |
| 32 | Q05086.4 UBE3A_HUMAN | LQFTTGTDRAPVGGL |
| 33 | Q5U5R9.2 HECD2_HUMAN | SNALMLLRPEEVEILVCGSPDLD |
| 34 | Q5U5R9.2 HECD2_HUMAN | LHFTTGSDRVPVGGM |
| 35 | O15033.3 AREL1_HUMAN | ENLLAIFDENELELLMCGTGDIS |
| 36 | O15033.3 AREL1_HUMAN | LQFTTGSSQLPPGGF |
| 37 | Q15751 HERC1_HUMAN | VPLLSLLTAKQLEQMVCGMPEIS |
| 38 | Q15751 HERC1_HUMAN | MRFVSGRSRLPANTA |
| 39 | O95714.2 HERC2_HUMAN | VPLLSLFTGYELETMVCGSPDIP |
| 40 | O95714.2 HERC2_HUMAN | LRFVWGRTRLP-RTI |
| 41 | Q5T447.1 HECTD3_HUMAN | QAVLDLLTWQELEKKVCGDPEVT |
| 42 | Q5T447.1 HECTD3_HUMAN | LRFVTGRSRLPARIY |
| 43 | Q15386.3 UBE3C_HUMAN | LEWLRMFDQQEIQVLISGAQVPI |
| 44 | Q15386.3 UBE3C_HUMAN | LKFVTSCSRPPLLGF |
| 45 | Q7Z3V4.3 UBE3B_HUMAN | PEWIRMFSTPELQRLISGDNAEI |
| 46 | Q7Z3V4.3 UBE3B_HUMAN | LKFVTSCSRPPLLGF |
| 47 | Q9ULT8.3 HECTD1_HUMAN | MEKLSSFSHEEVQMILCGNQSPS |
| 48 | Q9ULT8.3 HECTD1_HUMAN | LQFTTGCSTLPPGGL |
| 49 | O95071.2 UBR5_HUMAN | KNSLEDLTAEDFRLLVNGCGEVN |
| 50 | O95071.2 UBR5_HUMAN | VYFWTSSPSLPASEE |
| 51 | Q14669.1 TRIP12_HUMAN | LSHLQYFYPEELDQLLCGSKADT |
| 52 | Q14669.1 TRIP12_HUMAN | LQFVTGSPRLPVGGF |

1. An in silico method of identifying a small molecule allosteric inhibitor of a target protein family, comprising:
a. identifying a template protein member of the target protein family, the template protein member comprising a hinge domain, an allosteric binding site, and a catalytic site, the allosteric binding site different from the catalytic site, wherein binding of a small molecule allosteric inhibitor induces an allosteric change in the template protein member thereby inhibiting catalytic activity;
b. in silico modeling of the allosteric binding site of an inhibited structure of the template protein member by threading corresponding amino acid sequences of members of the target protein family onto the inhibited structure of the template protein member to create a model target protein;
c. in silico screening of a group of small molecules to identify small molecule allosteric inhibitor candidates predicted to bind to the allosteric binding site of the model target protein;
d. screening the small molecule allosteric inhibitor candidates to identify at least one small molecule allosteric inhibitor of the target protein family that induces an allosteric change in the target protein family upon binding to the allosteric binding site.

2. The method of claim 1, wherein the hinge domain is a glycine-hinge domain.

3. The method of claim 1, wherein the template protein member comprises an elongated αH10 traversing a conserved glycine residue, an N-lobe, a C-lobe, and a shortened hinge between the N-lobe and the C-lobe.

4. The method of claim 1, wherein the template protein member comprises a lock mechanism in an inhibited state enabled by an amino acid (AA) pair, the AA pair comprising a first AA and a second AA, the first AA disposed at the end of an elongated αH10, and the second AA disposed on the C-lobe, said AA pair creating a non-covalent bond between them.

5. The method of claim 4, wherein the AA pair comprises D636 and R686.

6. The method of claim 4, wherein the non-covalent bond is formed between an αH10 D636 and C-lobe R686 of the template protein member; and
wherein R686 is further stabilized by N507 of the template protein member, the N50 7allosterically shifted toward R686.

7. The method of claim 4, wherein the non-covalent bond is a pi bond or an electrostatic bond.

8. The method of claim 1, wherein the template protein member is a homologous to E6AP C-terminus (HECT) E3-ligase.

9. The method of claim 1, wherein the template protein member comprises a lock mechanism in an inhibited state.

10. The method of claim 1, wherein the small molecule allosteric inhibitor induces allosteric changes in the template protein member comprising elongation of an αH10 of the HECT E3-ligase over a glycine-hinge thereby restraining motion between C-lobes and N-lobes of the template protein member thereby inhibiting the template protein member.

11. The method of claim 1, wherein the template protein member is SMURF1.

12. The method of claim 1, wherein the allosteric binding site is a cryptic cavity.

13. An in silico method for predicting whether a HECT E3-ligase will undergo an allosteric change in response to binding a small molecule allosteric inhibitor, comprising:
a. Identifying a template HECT E3-ligase, the template HECT E3-ligase comprising a hinge domain, an allosteric binding site different from a catalytic site, wherein binding of a small molecule allosteric inhibitor induces an allosteric change in the template HECT E3-ligase thereby inhibiting catalytic activity;
b. Threading amino acid sequences of target HECT E3-ligases onto the inhibited structure of the template HECT E3-ligase to identify target HECT E3-ligases having homologous structure and amino acid sequence to the template HECT E3-ligase when inhibited by the small molecule allosteric inhibitor;
c. Identifying target HECT E3-ligases predicted to undergo allosteric changes upon small molecule allosteric inhibitor binding to the allosteric binding site.

14. The method of claim 13, wherein the hinge domain comprises a glycine-hinge domain.

15. The method of claim 13, wherein the target HECT E3-ligase is identified by having an elongated αH10 traversing a conserved glycine residue, an N-lobe, a C-lobe, and a shortened hinge between the N-lobe and the C-lobe when bound by the small molecule allosteric inhibitor.

16. The method of claim 13, wherein the template HECT E3-ligase comprises a lock mechanism in an inhibited state enabled by an amino acid (AA) pair, the AA pair comprising a first AA and a second AA, the first AA disposed at the end of an elongated αH10, and the second AA disposed on the C-lobe, said AA pair creating a non-covalent bond between them.

17. The method of claim 16, wherein the AA pair comprises D636 and R686.

18. The method of claim 16, wherein the non-covalent bond is a pi bond or an electrostatic bond.

19. The method of claim 16, wherein the target HECT E3-ligases are identified by having a non-covalent bond between αH10 D636 and C-lobe R686 when bound by the small molecule allosteric inhibitor; and
wherein R686 is further stabilized by N507 of the HECT E3-ligase, the N507 allosterically shifted toward R686 when bound by the small molecule allosteric inhibitor.

20. The method of claim 13, wherein the target HECT E3-ligase is identified when bound by the small molecule allosteric inhibitor by allosteric changes as compared to when unbound by the small molecule allosteric inhibitor, the allosteric changes comprising elongation of an αH10 and shortening of the hinge domain when bound by the small molecule allosteric inhibitor.

21. The method of claim 13, wherein the allosteric binding site is a cryptic cavity.

22. The method of claim 13, wherein the template HECT E3-ligase comprises a lock mechanism in an inhibited state.

23. The method of claim 13, wherein the template HECT E3-ligase is SMURF1.
